(19) United States
(12) Patent Application Publication
(10) Pub. No.: US 2021/0000727 A1
(43) Pub. Date: Jan. 7, 2021

(54) DITHIAZOCANE COMPOUNDS FOR THE COSMETIC USE THEREOF

(71) Applicant: L'OREAL, Paris (FR)

(72) Inventors: Xavier MARAT, AULNAY-SOUS-BOIS (FR); Amélie PREVOT-GUEGUINIAT, AULNAY-SOUS-BOIS (FR)

(21) Appl. No.: 16/955,745

(22) PCT Filed: Dec. 18, 2018
(86) PCT No.: PCT/EP2018/085620

§ 371 Date: Jun. 18, 2020

(30) Foreign Application Priority Data
Dec. 21, 2017 (FR) 1762851

(51) Int. Cl. A61K 8/49; A61K 8/60; A61Q 19/02; A61Q 5/08

(52) U.S. Cl. CPC A61K 8/4973; A61K 8/49; A61Q 5/08; A61Q 19/02; A61K 8/602

(57) ABSTRACT

The invention relates to the non-therapeutic cosmetic use of at least one compound of formula (I) as defined below, as an agent for bleaching, lightening and/or depigmenting keratin materials, especially the skin. The invention also relates to a non-therapeutic cosmetic process for depigmenting, lightening and/or bleaching keratin materials, especially the skin, using these compounds (I).

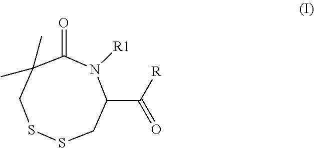

The present invention relates to the non-therapeutic cosmetic use of dithiazocane compounds, to compositions, especially cosmetic compositions, containing same and to the non-therapeutic cosmetic use thereof for depigmenting and/or bleaching keratin materials, in particular human skin. At various periods in their life, some people develop darker and/or more colored blemishes on their skin, and more especially on the hands, neck and the face, which give the skin heterogeneity. These blemishes are especially due to a high concentration of melanin in the keratinocytes located at the surface of the skin.

The use of highly effective inoffensive topical depigmenting substances is most particularly sought for the purpose of reducing and/or treating pigmentary blemishes.

The mechanism of formation of the pigmentation of the skin, i.e. of the formation of melanin, is particularly complex and involves, schematically, the following main steps:

Tyrosine→Dopa→Dopaquinone→Dopachrome→Melanin

Tyrosinase (monophenol dihydroxyl phenylalanine: oxygen oxidoreductase EC 1.14.18.1) is the essential enzyme involved in this sequence of reactions. In particular, it catalyzes the conversion reaction of tyrosine to give dopa (dihydroxyphenylalanine), by virtue of its hydroxylase activity, and the conversion reaction of dopa to give dopaquinone, by virtue of its oxidase activity. This tyrosinase acts only when it is in mature form under the effect of certain biological factors.

A substance is acknowledged as being depigmenting if it acts directly on the vitality of the epidermal melanocytes where melanogenesis takes place, and/or if it interferes with one of the steps of melanin biosynthesis, either by inhibiting one of the enzymes involved in melanogenesis, or by inserting itself as a structural analog of one of the chemical compounds of the melanin synthesis chain, which chain can then be blocked, thus ensuring depigmentation.

Arbutin and kojic acid are known as skin depigmenting agents.

There remains a need for a novel agent for bleaching keratin materials, in particular human skin, which is effective and does not have the drawbacks of the depigmenting agents known from the prior art, that is to say which is non-toxic and/or non-allergenic for keratin materials, in particular the skin, while also being stable in a composition, especially a novel agent which is stable (for example chemical stability or photostability), and especially having an odor and/or a color which is stable over time, or else alternatively which has a reinforced action so as to be able to be used in a smaller amount, which considerably reduces the side effects observed.

Cyclic disulfide derivative compounds are known in the prior art for the pharmaceutical use thereof as medicaments of use for treating cataracts, ischemic cardiac diseases and the like, caused by oxidative stress, especially in document WO 01/64661. However, the use of these compounds to bleach keratin materials, especially human skin, is not suggested by this document.

In this regard, the Applicant has discovered, surprisingly and unexpectedly, that certain dithiazocane compounds have good depigmenting activity, even at low concentration, without showing any cytotoxicity.

The invention relates to the non-therapeutic cosmetic use of at least one compound of formula (I) or of a composition containing at least one compound of formula (I) as defined below, as an agent for bleaching, lightening and/or depigmenting keratin materials, especially human skin.

Similarly, a subject of the invention is a composition comprising, in a physiologically acceptable medium, at least one compound of formula (I) as defined below.

Another subject of the invention is a non-therapeutic cosmetic treatment process for depigmenting, lightening and/or bleaching keratin materials, especially human skin, comprising the application to the keratin materials, and especially human skin, of at least one compound of formula (I) as defined below or of a composition containing same.

In another variant, one subject of the invention is some compounds of formula (I) as defined below for the dermatological use thereof for depigmenting the skin.

The compounds of formula (I) in accordance with the invention, as defined below, make it possible to depigment and/or lighten efficiently, or even bleach, human skin. They are especially intended to be applied to the skin of individuals exhibiting brownish pigmentation blemishes or liver spots, or to the skin of individuals wishing to reduce and/or soften the appearance of a brownish color caused by melanogenesis.

For the purposes of the present invention, the term “keratin materials” is intended to mean human keratin materials, and in particular the skin.

More particularly, “keratin materials” denote human skin, and even more particularly the skin of the face, neck and hands.

Therefore, a subject of the invention is the non-therapeutic cosmetic use of at least one compound of formula (I) or of a composition containing at least one compound of formula (I) as an agent for bleaching, lightening and/or depigmenting keratin materials, especially human skin, said compounds corresponding to the following formula (I):

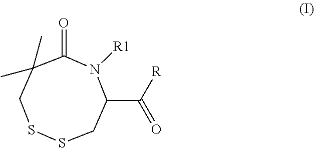

in which:

R denotes a radical OR′ or NRₐR_{b};

R1 denotes a hydrogen atom or a (C₁-C₁₈) alkyl group;

R′ represents:

i) a hydrogen atom;

ii) a (C₁-C₁₈)alkyl group, said alkyl group being
a) optionally substituted, especially with one or more identical or different radicals chosen from
   hydroxyl;
   (hetero)cycloalkyl, optionally substituted especially with one or more identical or different radicals chosen from hydroxyl, C₁-C₄ alkyl, C₁-C₄ hydroxyalkyl, C₁-C₄ alkoxy and/or at least one of the ring members of said (hetero)cycloalkyl denoting a ketone (—CO—);
   aryl, optionally substituted especially with one or more identical or different radicals chosen from C₁-C₄ alkyl, C₁-C₄ alkoxy, hydroxyl,

and/or said alkyl radical being
b) optionally interrupted with one or more heteroatoms such as O, S and N(R₅), with R₅ representing a hydrogen atom or a (C₁-C₄)alkyl group such as methyl;

iii) a (C₂-C₁₈)alkenyl group, said alkenyl radical being
a) optionally substituted, especially with one or more identical or different radicals chosen from
   hydroxyl,
   aryl, optionally substituted especially with one or more identical or different radicals chosen from C₁-C₄ alkyl, C₁-C₄ alkoxy, hydroxyl,
      and/or said alkenyl radical being
b) optionally interrupted with one or more heteroatoms such as O, S and N(R₅), with R₅ representing a hydrogen atom or a (C₁-C₄)alkyl group such as methyl;

iv) an optionally substituted aryl or heteroaryl radical;

Ra and Rb independently represent:
i) a hydrogen atom;
ii) a (C₁-C₁₈)alkyl group, said alkyl group being
   a) optionally substituted, especially with one or more identical or different radicals chosen from
      hydroxyl,
      carboxy,
      (C₁-C₆)alkyloxycarbonyl,
      aryl, optionally substituted especially with one or more identical or different radicals chosen from C₁-C₄ alkyl, C₁-C₄ alkoxy, hydroxyl,
and/or said alkyl radical being
   b) optionally interrupted with one or more heteroatoms such as O, S and N(R₅), with R₅ representing a hydrogen atom or a (C₁-C₄)alkyl group such as methyl;
iii) a (C₂-C₁)alkenyl group, said alkenyl group being
   a) optionally substituted, especially with one or more identical or different radicals chosen from
      hydroxyl,
      carboxy,
      (C₁-C₆)alkyloxycarbonyl,
      aryl, optionally substituted especially with one or more identical or different radicals chosen from C₁-C₄ alkyl, C₁-C₄ alkoxy, hydroxyl,
and/or said alkenyl radical being
   b) optionally interrupted with one or more heteroatoms such as O, S and N(R₅), with R₅ representing a hydrogen atom or a (C₁-C₄)alkyl group such as methyl;
iv) an aryl or heteroaryl radical, optionally substituted especially with one or more identical or different radicals chosen from C₁-C₄ alkyl, hydroxyl, C₁-C₄ alkoxy, such as methoxy;
v) a heterocycloalkyl radical, optionally substituted especially with one or more identical or different radicals chosen from hydroxyl, C₁-C₄ hydroxyalkyl, C₁-C₄ alkyl, C₁-C₄ alkoxy and/or at least one of the ring members of said heterocycloalkyl radical denoting a ketone (—CO—);

or else Ra and Rb form, together with the nitrogen atom to which they are attached, an optionally substituted heterocycloalkyl, such as piperazino, piperidino or morpholino;

and also the salts thereof, the solvates thereof and the optical and/or geometrical isomers thereof, including enantiomers and diastereoisomers, and the racemates thereof, alone or in a mixture.

The asterisk “*” denotes the point of attachment of the radical to the rest of the compound.

For the purposes of the present invention, the term “compounds of formula (I)”, or “compounds 1′ to 15′ and 18′ to 20′, in particular compounds 1 and 2”, is intended to mean the compounds as defined in the present description, and also the solvates thereof such as hydrates, the optical and geometrical isomers thereof, the tautomers thereof, and the organic or mineral base or acid salts thereof.

The acceptable solvates of the compounds used in the present invention comprise conventional solvates such as those formed during the last step of the preparation of said compounds due to the presence of solvents. Mention may be made, by way of example, of the solvates due to the presence of water (hydrates) or of linear or branched alcohols, such as ethanol or isopropanol.

The salts of the compounds (I) which comprise at least one acid function can be chosen from metal salts, for example aluminum (Al³⁺), zinc (Zn²⁺), manganese (Mn²⁺) or copper (Cu²⁺); alkali metal salts, for example lithium (Li⁺), sodium (Na⁺) or potassium (K+); or alkaline-earth metal salts, for example calcium (Ca²⁺) or magnesium (Mg²⁺). They can also be ammonium derivatives of formula NH₄⁺ or organic salts such as ammoniums of formula Y₃NH⁺, NY₃ denoting an organic amine, the Y radicals being identical or different, it being possible for two or three Y radicals to form, in pairs, a ring with the nitrogen atom which carries them or it being possible for NY₃ to denote an aromatic amine. The organic amines are for example alkylamines, for instance methylamine, dimethylamine, trimethylamine, triethylamine or ethylamine, or hydroxyalkylamines, for instance 2-hydroxyethylamine, bis(2-hydroxyethyl)amine or tris(2-hydroxyethyl)amine, or cycloalkylamines, for instance bicyclohexylamine or glucamine, piperidine, or pyridines and the like, for example collidine, quinine or quinoline, or amino acids which are basic in nature, for instance lysine or arginine.

The salts of the compounds of formula (I) which comprise at least one amine function can also be salts of an organic acid such as citric acid, lactic acid, tartaric acid, aspartic acid, glutamic acid, acetic acid, formic acid, trifluoroacetic acid, hydrochloric acid, glycolic acid or malic acid.

In the case where the compounds according to the invention are in salt form, the cations are of course in an amount which ensures the electro-neutrality of the compounds of formula (I).

The salts of the compounds of formula (I) according to the invention which comprise at least one acid function can advantageously be chosen from the metal salts Cu²⁺, Mn²⁺ and Zn²⁺, the alkali metal salts Li⁺, Na⁺ and K⁺ and the alkaline-earth metal salts Ca²⁺ and Mg²⁺.

According to another variant, the salts of the compounds of formula (I) according to the invention which comprise at least one acid function can advantageously be chosen from ammoniums, preferably from the salts of amino acids which are basic in nature, for instance lysine or arginine or from diethanolamine salts or triethanolamine salts.

For the purposes of the present invention and unless otherwise indicated:
the saturated or unsaturated and optionally fused rings may also be optionally substituted;
the “alkyl” radicals are saturated, linear or branched, generally C₁-C₁₈, particularly C₁-C₁₀, hydrocarbon-based radicals, preferably C₁-C₆ alkyl radicals; as C₁-C₁₄ alkyl group, mention may especially be made of methyl, ethyl, isopropyl, n-propyl, n-butyl, t-butyl, isobutyl, sec-butyl, pentyl, isopentyl, neopentyl, n-hexyl, n-heptyl and n-octyl groups;
the “alkenyl” radicals are linear or branched, unsaturated C₂-C₁₈, especially C₂-C₁₀ and more particularly C₂-C₆ hydrocarbon-based radicals, comprising one or more conjugated or unconjugated double bonds, such as ethylene, propylene, butylene, pentylene, 2-methylpropylene, prenyl and decylene, in particular propylene;
the “aryl” radicals are monocyclic or polycyclic, fused or unfused carbon-based radicals, preferentially comprising from 6 to 30 carbon atoms and at least one ring of which is aromatic; preferentially, the aryl radical is chosen from a phenyl, a biphenyl, a naphthyl, an indenyl, an anthracenyl, and a tetrahydronaphthyl; more preferentially, aryl denotes phenyl; the aryl radicals may be substituted especially with one or more identical or different radicals chosen from hydroxyl, (C₁-C₆) and more particularly (C₁-C₄) alkyl, (C₁-C₆) and more particularly (C₁-C₄) alkoxy, carboxy, (C₁-C₄)alkyloxycarbonyl;
the “alkoxy” radicals are alkyloxy radicals with alkyl as previously defined, the alkyl part of the alkoxy generally being C₁-C₁₈, preferably C₁-C₁₀, more preferentially C₁-C₄, such as methoxy, ethoxy, propoxy and butoxy; when mention is made of unsaturated, this implies that the alkoxy group can represent an alkenyloxy group with alkenyl as previously defined;
the “cycloalkyl” radicals are saturated or partially unsaturated, non-aromatic C₄-C₈ cycloalkyl (cyclic alkyl) radicals, preferably cyclopentyl and cyclohexyl radicals; the cycloalkyl radicals may be substituted cycloalkyl radicals, in particular substituted with alkyl, alkoxy, carboxylic acid, hydroxyl or amine groups, and/or at least one of the ring members thereof may be a ketone;
the “heterocycloalkyl” radicals are saturated or partially unsaturated, non-aromatic heterocyclic radicals comprising from 4 to 8 ring members, which comprise from 1 to 3 heteroatoms, especially chosen from oxygen, sulfur and nitrogen, preferably the morpholino, piperazino and piperidino, tetrahydrofuran, tetrahydropyran radicals, preferably tetrahydrofuran or tetrahydropyran; the heterocycloalkyl radicals may be substituted radicals, in particular substituted with alkyl, alkoxy, carboxylic acid, hydroxyl, amine and ketone groups, especially with hydroxyl or ketone groups;
the “aryl” or “heteroaryl” radicals may be substituted with at least one radical chosen from:

i) (C₁-C₁₀), preferably C₁-C₈ and more preferentially C₁-C₆ alkyl, such as C₁-C₄ alkyl, said alkyl radical being optionally substituted with one or more radicals chosen from hydroxyl, optionally unsaturated (C₁-C₄)alkoxy, (poly)hydroxy(C₂-C₄)alkoxy, acylamino, amino substituted with two identical or different C₁-C₄ alkyl radicals, optionally bearing at least one hydroxyl group, or it being possible for the two radicals to form, with the nitrogen atom to which they are attached, a saturated or unsaturated heterocycle comprising from 5 to 7 ring members, preferably 5 or 6 ring members, said heterocycle being optionally substituted and/or optionally comprising another heteroatom identical to or different from nitrogen;

ii) halogen;

iii) hydroxyl;

iv) alkoxy, especially C₁-C₄ alkoxy such as methoxy;

v) C₁-C₁₀ alkoxycarbonyl, especially C₁-C₄ alkoxycarbonyl such as methoxycarbonyl or ethoxycarbonyl;

vi) C₂-C₄ (poly)hydroxyalkoxy;

vii) C₂-C₄ alkylcarbonyloxy, preferentially —O-acetyl or acetyloxy;

viii) 5- or 6-membered heterocycloalkyl;

ix) 5- or 6-membered heteroaryl, optionally substituted with a (C₁-C₄)alkyl radical, preferentially methyl;
the “alkyl” or “alkenyl” or “(hetero)cycloalkyl” radicals may be substituted with at least one radical chosen from:

i) halogen;

ii) hydroxyl;

iii) alkoxy, especially C₁-C₄ alkoxy such as methoxy;

iv) C₁-C₁₀ alkoxycarbonyl, especially C₁-C₄ alkoxycarbonyl such as methoxycarbonyl or ethoxycarbonyl;

v) C₂-C₄ (poly)hydroxyalkoxy;

vi) C₂-C₄ alkylcarbonyloxy, preferentially —O-acetyl or acetyloxy;

vii) 5- or 6-membered heterocycloalkyl;

viii) 5- or 6-membered heteroaryl, optionally substituted with a (C₁-C₄)alkyl radical, preferentially methyl;
the “heteroaryl” radicals are radicals comprising, in at least one ring, one or more heteroatoms chosen in particular from O, N and S, preferably O or N, optionally substituted in particular with one or more alkyl, alkoxy, carboxy, hydroxyl, amine or ketone groups, and at least one ring of which is aromatic. These rings may contain one or more oxo groups on the carbon atoms of heteroaryl; mention may especially be made, among the heteroaryl radicals that may be used, of furyl, pyranyl, pyrrolyl, imidazolyl, pyrazolyl, pyridyl, thienyl, and pyrimidinyl groups; optionally, the heteroaryl groups are fused groups, such as benzofuranyl, chromenyl, xanthenyl, indolyl, isoindolyl, quinolyl, isoquinolyl, chromanyl, isochromanyl, indolinyl, isoindolinyl, coumarinyl or isocoumarinyl groups, it being possible for these groups to be substituted, in particular with one or more OH groups,
a (hetero)aryl radical denotes an aryl radical or a heteroaryl radical;
a (hetero)cycloalkyl radical denotes a heterocycloalkyl radical or a cycloalkyl radical.

According to a particular embodiment, the compounds of formula (I) are of R configuration. In other words, the configuration of the carbon of the dithiazocane ring bearing the —CO—R radical is the R configuration.

According to a particular embodiment, the compounds of formula (I) are such that R₁ denotes a (C₁-C₆)alkyl group such as methyl or a hydrogen atom, preferably a hydrogen atom.

According to a particular embodiment, the compounds of formula (I) are such that R denotes an OR′ radical, R′ being as defined previously.

According to a particular embodiment, the compounds of formula (I) are such that R denotes a radical OR′ in which R′ denotes i) a hydrogen atom or ii) a (C₁-C₁₈)alkyl group, in particular i) a hydrogen atom or ii) a (C₁-C₄)alkyl group such as methyl, ethyl, n-propyl, isopropyl, n-butyl or isobutyl.

According to a particular embodiment, the compounds of formula (I) are such that R denotes a radical OR′ in which R′ denotes ii) a (C₁-C₁)alkyl group, in particular a (C₁-C₆)alkyl group, optionally substituted with one or more identical or different radicals chosen from
hydroxyl;
heterocycloalkyl substituted with one or more identical or different radicals chosen from hydroxyl, C₁-C₄ alkyl, C₁-C₄ hydroxyalkyl, C₁-C₄ alkoxy and/or one of the ring members of said heterocycloalkyl denotes a ketone (—CO—); preferably substituted with one or more identical or different radicals chosen from hydroxyl, C₁-C₄ hydroxyalkyl and/or one of the ring members of said heterocycloalkyl denotes a ketone (—CO—);
phenyl, optionally substituted with one or more identical or different radicals chosen from C₁-C₄ alkyl, C₁-C₄ alkoxy, hydroxyl, preferably phenyl substituted with one or more identical or different radicals chosen from hydroxyl or C₁-C₄ alkoxy, such as methoxy.

According to a particular embodiment, the compounds of formula (I) are such that R denotes a radical OR′ in which R′ denotes ii) a (C₁-C₁₈) alkyl group such as (C₁-C₁₄)alkyl, said alkyl group being interrupted with one or more heteroatoms such as O, S and N(R₅), with R₅ representing a hydrogen atom or a (C₁-C₄)alkyl group such as methyl; preferably interrupted with one or more non-adjacent oxygen atoms.

According to a particular embodiment, the compounds of formula (I) are such that R denotes a radical OR′ in which R′ denotes a radical of formula (B′1):

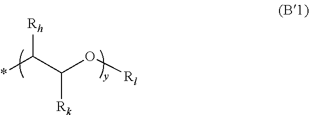

in which Rₕ and Rₖ independently of one another denote a hydrogen atom or a methyl radical, it being understood that R and Rₖ preferably cannot simultaneously denote a methyl radical; Rₗ denotes H or OH; y denotes an integer from 1 to 10 inclusive (limit values included).

According to a particular embodiment, the compounds of formula (I) are such that R denotes a radical OR′ in which R′ denotes ii) a (C₁-C₁₈)alkenyl group, in particular a (C₁-C₆)alkenyl group, optionally substituted with one or more identical or different radicals chosen from
hydroxyl;
heterocycloalkyl substituted with one or more identical or different radicals chosen from hydroxyl, C₁-C₄ alkyl, C₁-C₄ hydroxyalkyl, C₁-C₄ alkoxy and/or one of the ring members of said heterocycloalkyl denotes a ketone (—CO—); preferably substituted with one or more identical or different radicals chosen from hydroxyl, C₁-C₄ hydroxyalkyl and/or one of the ring members of said heterocycloalkyl denotes a ketone (—CO—);
phenyl, optionally substituted with one or more identical or different radicals chosen from C₁-C₄ alkyl, C₁-C₄ alkoxy, hydroxyl, preferably phenyl substituted with one or more identical or different radicals chosen from hydroxyl or C₁-C₄ alkoxy, such as methoxy;
   said alkenyl radical preferably being substituted with a phenyl substituted with one or more identical or different radicals chosen from hydroxyl or C₁-C₄ alkoxy, such as methoxy.

According to a particular embodiment, the compounds of formula (I) are such that R denotes a radical NRₐR_{b}, in which Rₐ and R_{b} are as defined previously.

According to a particular embodiment, the compounds of formula (I) are such that R denotes a radical NRₐR_{b}, in which Rₐ and R_{b} independently represent:
i) a hydrogen atom;
ii) a (C₁-C₁₈)alkyl group, in particular (C₁-C₈)alkyl group such as methyl or n-heptyl.

According to another particular embodiment, the compounds of formula (I) are such that R denotes a radical NRₐR_{b}, in which Rₐ denotes a hydrogen atom and R_{b} represents ii) a (C₁-C₁₈)alkyl, in particular (C₁-C₄)alkyl group such as methyl, said alkyl group being optionally substituted with one or more identical or different radicals chosen from
hydroxyl,
carboxy,
(C₁-C₆)alkyloxycarbonyl,
phenyl, optionally substituted especially with one or more identical or different radicals chosen from C₁-C₄ alkyl, C₁-C₄ alkoxy, hydroxyl.

According to another particular embodiment, the compounds of formula (I) are such that R denotes a radical NRₐR_{b}, in which Rₐ denotes a hydrogen atom and R_{b} represents a radical of the following formula B′2:

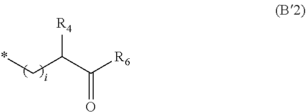

in which:
i=0 or 1;
R₄ represents a hydrogen atom or a (C₁-C₆)alkyl group optionally substituted with one or more groups chosen from i) —Z—C(Z′)—Z″—R_{C} with Z, Z′, and Z″, which are identical or different, representing an oxygen or sulfur atom, and N(R_{D}), R_{C} and R_{D}, which are identical or different, representing a hydrogen atom or a (C₁-C₄)alkyl group; preferably, Z, Z′ and Z″ represent an N(R_{D}) group such as NH; ii) (hetero)aryl such as imidazolyl, indolyl, phenyl, optionally substituted especially with a hydroxyl group; iii) (di)(C₁-C₄)(alkyl)amino, iv) —C(Z′)—Z″—R_{C}, with Z′, Z″ and R_{C} as defined above, in particular Z′ represents an oxygen atom, Z″ represents an oxygen atom or an N(R_{D}) group such as NH, and R_{C} represents a hydrogen atom; v) —Z′″—R_{C}, with Z′″ representing an oxygen, sulfur or selenium atom or an NH group and R_{C} is as defined previously; in particular, R₄ represents a (C₁-C₆)alkyl group optionally substituted with i) —Z—C(Z′)—Z″—R_{C}, with Z, Z′, and Z″, which are identical or different, representing an oxygen or sulfur atom, and N(R_{D}), R_{C} and R_{D}, which are identical or different, representing a hydrogen atom or a (C₁-C₄)alkyl group; preferably Z, Z′ and Z″ represent an N(R_{D}) group such as NH;
   or, when i=0, Rₐ and R₄ form, together with the nitrogen atom which bears Rₐ and with the carbon atom which bears R₄, a saturated 5- or 6-membered, preferably 5-membered, heterocycle such as a pyrrolidine ring;
R₆ denotes:
   i) a hydroxyl radical —OH;
   ii) a saturated or unsaturated (C₁-C₆)alkoxy radical; or
   iii) an —NRfRg radical, with Rf and Rg, which are identical or different, representing a hydrogen atom, or a (C₁-C₆)alkyl group; preferably, R₆ denotes a hydroxyl radical or a saturated or unsaturated (C₁-C₆) alkoxy radical such as methyl or ethyl.

According to another particular embodiment, the compounds of formula (I) are such that R denotes a radical NRₐR_{b} in which Rₐ denotes a hydrogen atom and R_{b} represents a radical of formula (B′2) as defined above, in which formula (B′2) R₆ denotes a hydroxyl radical or a saturated or unsaturated (C₁-C₆)alkoxy radical such as methyl or ethyl, and R₄ represents a hydrogen atom or R₄ is chosen from radicals (a1) to (a32) described below:

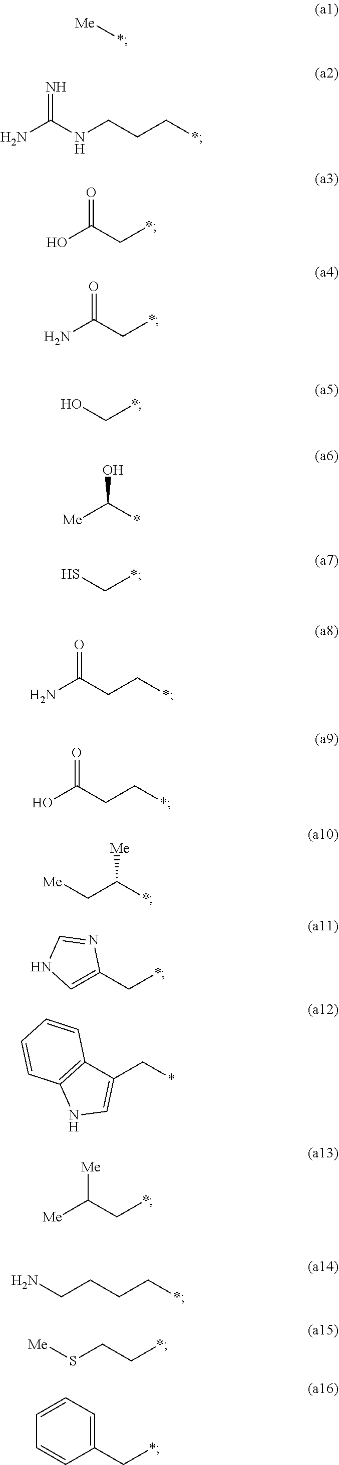

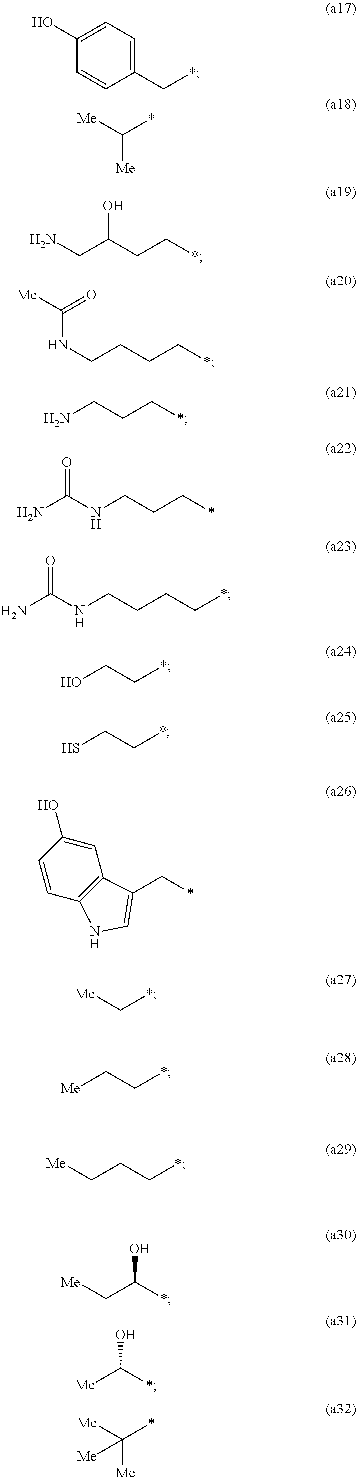

or, when i=0, Rₐ and R₄ form, together with the nitrogen atom which bears Rₐ and with the carbon atom which bears R₄, a saturated 5- or 6-membered heterocycle of formula A1, A2 or A3 below, preferably 5-membered, such as a pyrrolidine ring A2;

a saturated heterocycle of formula A1 or A2 or A3:

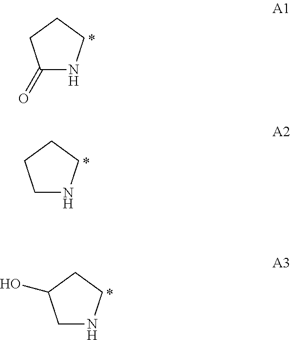 [SMILES: O=C1CCCN1 ; C1CCNC1 ; OC1CCNC1]

According to another particular embodiment, the compounds of formula (I) are such that R denotes a radical NRₐR_{b}, in which Rₐ denotes a hydrogen atom and R_{b} represents a heterocycloalkyl radical, optionally substituted especially with one or more identical or different radicals chosen from hydroxyl, C₁-C₄ hydroxyalkyl, C₁-C₄ alkyl, C₁-C₄ alkoxy, and/or at least one of the ring members of said heterocycloalkyl radical denoting a ketone (—CO—); preferably, the heterocycloalkyl radical is a glucopyran.

According to another particular embodiment, the compounds of formula (I) are such that R denotes a radical NRₐR_{b}, in which Rₐ denotes a hydrogen atom and R_{b} represents a glucopyran radical of formula (V)

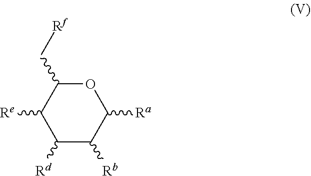

in which formula (V) R^{a}, R^{b}, R^{d}, R^{e} and R^{f}, which are identical or different, represent i) a hydroxyl group, ii) (C₁-C₄)alkoxy, the alkyl part of which may optionally be substituted, especially with one or more hydroxyl groups, iii) carboxy, and iv) an NR₁R₂ group with R₁ and R₂, which are identical or different, chosen from a hydrogen atom, (C₁-C₄)alkyl and acetyl;
it being understood that one of the radicals R^{a}, R^{b}, R^{d}, R^{e} and R^{f} represents a covalent bond with the nitrogen atom of the NRₐR_{b} radical; the configuration of said compounds of formula (V) being D or L, preferably D, and of a (alpha) or β (beta) anomeric configuration.

Preferably, the compounds of formula (I) and also the salts thereof, the solvates thereof and the optical and/or geometrical isomers thereof, including enantiomers and diastereoisomers, and the racemates thereof, alone or in a mixture, are such that:

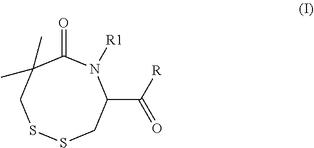

R denotes a radical OR′ or NRₐR_{b};
R₁ denotes a hydrogen atom or a (C₁-C₆)alkyl group such as methyl; preferably a hydrogen atom;
R′ represents:

i) a hydrogen atom; or

ii) a (C₁-C₈)alkyl, in particular (C₁-C₆)alkyl group, said alkyl group being optionally substituted with one or more identical or different radicals chosen from:
hydroxyl;
heterocycloalkyl substituted with one or more identical or different radicals chosen from hydroxyl, C₁-C₄ alkyl, C₁-C₄ hydroxyalkyl, C₁-C₄ alkoxy and/or one of the ring members of said heterocycloalkyl denotes a ketone (—CO—); preferably substituted with one or more identical or different radicals chosen from hydroxyl, C₁-C₄ hydroxyalkyl and/or one of the ring members of said heterocycloalkyl denotes a ketone (—CO—);
phenyl, optionally substituted with one or more identical or different radicals chosen from C₁-C₄ alkyl, C₁-C₄ alkoxy, hydroxyl, preferably phenyl substituted with one or more identical or different radicals chosen from hydroxyl or C₁-C₄ alkoxy, such as methoxy; or

iii) a (C₁-C₁)alkyl group such as (C₁-C₁₄)alkyl, said alkyl group being interrupted with one or more heteroatoms such as O, S and N(R₅), with R₅ representing a hydrogen atom or a (C₁-C₄)alkyl group such as methyl; preferably interrupted with one or more non-adjacent oxygen atoms; or

iv) a (C₁-C₁₈)alkenyl group, in particular (C₁-C₆)alkenyl, optionally substituted with one or more identical or different radicals chosen from
hydroxyl;
heterocycloalkyl substituted with one or more identical or different radicals chosen from hydroxyl, C₁-C₄ alkoxy, C₁-C₄ hydroxyalkyl, C₁-C₄ alkoxy and/or one of the ring members of said heterocycloalkyl denotes a ketone (—CO—); preferably substituted with one or more identical or different radicals chosen from hydroxyl, C₁-C₄ hydroxyalkyl and/or one of the ring members of said heterocycloalkyl denotes a ketone (—CO—);
phenyl, optionally substituted with one or more identical or different radicals chosen from C₁-C₄ alkyl, C₁-C₄ alkoxy, hydroxyl, preferably phenyl substituted with one or more identical or different radicals chosen from hydroxyl or C₁-C₄ alkoxy, such as methoxy;
   said alkenyl radical preferably being substituted with a phenyl substituted with one or more identical or different radicals chosen from hydroxyl or C₁-C₄ alkoxy, such as methoxy;
   Rₐ represents:
i) a hydrogen atom;
ii) a (C₁-C₁₈)alkyl group, in particular (C₁-C₈)alkyl group such as methyl or n-heptyl;
   R_{b} represents:

i) a (C₁-C₁₈)alkyl, in particular (C₁-C₄)alkyl group such as methyl, said alkyl group being optionally substituted with one or more identical or different radicals chosen from
hydroxyl,
carboxy,
(C₁-C₆)alkyloxycarbonyl,
phenyl, optionally substituted especially with one or more identical or different radicals chosen from C₁-C₄ alkyl, C₁-C₄ alkoxy, hydroxyl;

ii) a (C₂-C₁₈)alkenyl, in particular (C₂-C₄)alkenyl group, said alkenyl group being optionally substituted with one or more identical or different radicals chosen from
hydroxyl,
carboxy,
(C₁-C₆)alkyloxycarbonyl,
phenyl, optionally substituted especially with one or more identical or different radicals chosen from C₁-C₄ alkyl, C₁-C₄ alkoxy, hydroxyl;

iii) a heterocycloalkyl radical, optionally substituted especially with one or more identical or different radicals chosen from hydroxyl, C₁-C₄ hydroxyalkyl, C₁-C₄ alkyl, C₁-C₄ alkoxy and/or at least one of the ring members of said heterocycloalkyl radical denoting a ketone (—CO—); preferably a radical of formula (V) as defined previously;

or else Rₐ and R_{b} form, together with the nitrogen atom to which they are attached, an optionally substituted heterocycloalkyl, such as piperazino, piperidino or morpholino;
and also the salts thereof, the solvates thereof and the optical and/or geometrical isomers thereof, including enantiomers and diastereoisomers, and the racemates thereof, alone or in a mixture.

Preferably, the compounds of formula (I) are chosen from the compounds 1′ to 15′ and from the compounds 18′ to 20′ below and also the salts thereof, the solvates thereof and the optical and/or geometrical isomers thereof, including enantiomers and diastereoisomers, and the racemates thereof, alone or in a mixture:

| Structure | Compound |
|---|---|
| 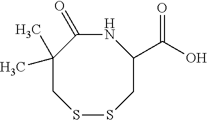 | 1′ |
| 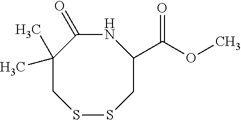 | 2′ |
| 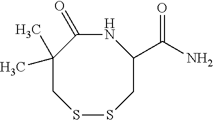 | 3′ |
| 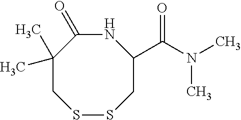 | 4′ |
| 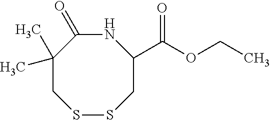 [SMILES: CCOC(=O)C1CSSCC(C)(C)C(=O)N1] | 5′ |
| 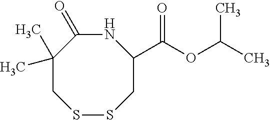 [SMILES: CC(C)OC(=O)C1CSSCC(C)(C)C(=O)N1] | 6′ |
| 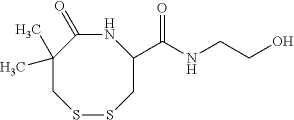 [SMILES: CC1(C)CSSCC(C(=O)NCCO)NC1=O] | 7′ |
| 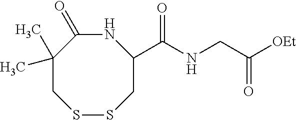 [SMILES: CCOC(=O)CNC(=O)C1CSSCC(C)(C)C(=O)N1] | 8′ |
| 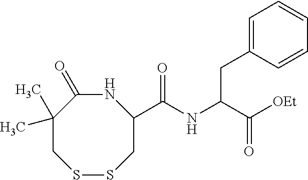 [SMILES: CCOC(=O)C(Cc1ccccc1)NC(=O)C1CSSCC(C)(C)C(=O)N1] | 9′ |
| 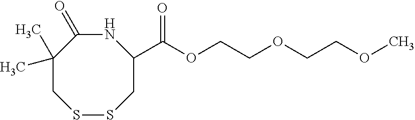 | 10′ |
| 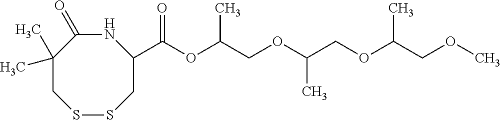 | 11′ |
| 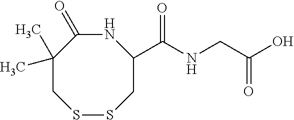 [SMILES: CC1(C)CSSCC(C(=O)NCC(=O)O)NC1=O] | 12′ |
| 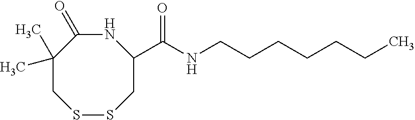 [SMILES: CCCCCCCNC(=O)C1CSSCC(C)(C)C(=O)N1] | 13′ |
| 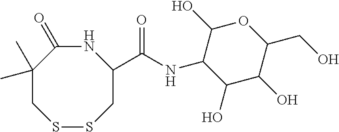 [SMILES: CC1(C)CSSCC(C(=O)NC2C(O)OC(CO)C(O)C2O)NC1=O] | 14′ |
| 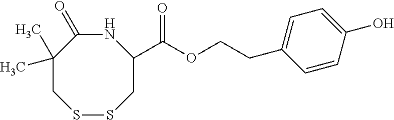 [SMILES: CC1(C)CSSCC(C(=O)OCCc2ccc(O)cc2)NC1=O] | 15′ |
| 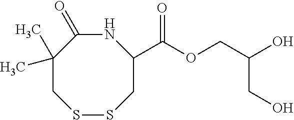 [SMILES: CC1(C)CSSCC(C(=O)OCC(O)CO)NC1=O] | 18′ |
| 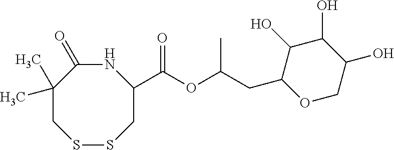 [SMILES: CC(CC1OCC(O)C(O)C1O)OC(=O)C1CSSCC(C)(C)C(=O)N1] | 19′ |
| 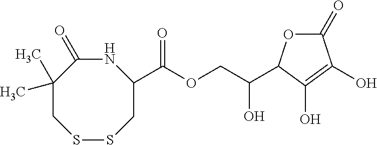 [SMILES: CC1(C)CSSCC(C(=O)OCC(O)C2OC(=O)C(O)=C2O)NC1=O] | 20′ |

Even more preferentially, the compounds of formula (I) are chosen from the compounds 1 to 4 below and also the salts thereof, the solvates thereof, alone or in a mixture, and/or from the compounds 5′ to 15′, and from compounds 18′ to 20′ described above and also the salts thereof, the solvates thereof, and the optical and/or geometrical isomers thereof, including enantiomers and diastereoisomers, and the racemates thereof, alone or in a mixture:

| | Chemical | Com- |
|---|---|---|
| Structure | name | pound |
| 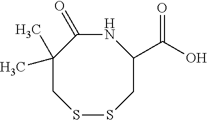 | 7,7-dimethyl-6-oxo- 1,2,5-dithiazocane-4- carboxylic acid | 1 (R enan- tiomer) |
| 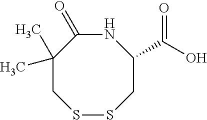 | CAS 128620-82-6 for the R isomer | |
| 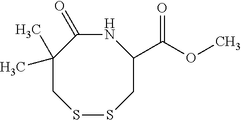 | methyl 7,7-dimethyl- 6-oxo-1,2,5- dithiazocane-4- carboxylate | 2 |
| 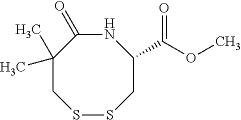 | CAS 128638-96-0 for the R isomer | |
| 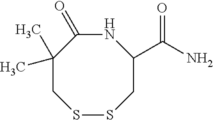 | 7,7-dimethyl-6-oxo- 1,2,5-dithiazocane-4- carboxamide | 3 |
| 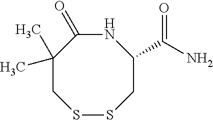 | CAS 358678-52-1 for the R isomer | |
| 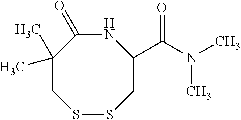 | N,N,7,7-tetramethyl- 6-oxo-1,2,5- dithiazocane- 4-carboxamide | 4 |
| 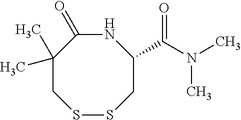 | CAS 358678-56-5 for the R isomer | |

The invention relates to the non-therapeutic cosmetic use of at least one compound of formula (I) as defined previously or of a composition containing at least one compound of formula (I), and more particularly of at least one compound chosen from the compounds 1′ to 15′ and from the compounds 18′ to 20′ as described previously, more particularly chosen from the compounds 1 and 2 described above, and even more preferentially the compound 1, as an agent for bleaching, lightening and/or depigmenting keratin materials, especially the skin.

### Novel Compounds

Some compounds of formula (I) are novel and constitute another subject of the invention.

Thus, another subject of the invention is the compounds of formula (I) as defined above, with the exception of the following compounds Cp1 to Cp5:

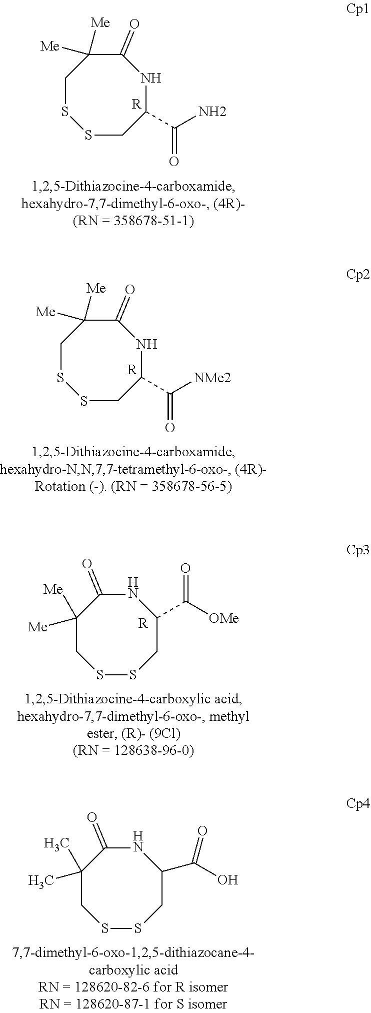

and also the salts thereof, the solvates thereof and the optical and/or geometrical isomers thereof, including enantiomers and diastereoisomers, and the racemates thereof, alone or in a mixture.

Thus, another subject of the invention relates to the compounds of formula (F1) which are novel.

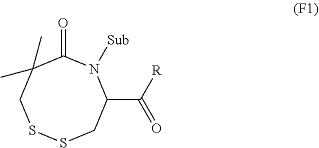

in which:

R is as defined previously for the compounds of formula (I);

Sub denotes a (C₁-C₁₈)alkyl group such as methyl;

and also the salts thereof, the solvates thereof and the optical and/or geometrical isomers thereof, including enantiomers and diastereoisomers, and the racemates thereof, alone or in a mixture.

Another subject of the invention relates to the compounds of formula (F2) which are also novel compounds:

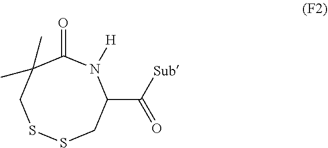

in which formula (F2):

Sub′ denotes a radical OT′, or NTₐT_{b};

T′ represents:

i) a (C₂-C₁)alkyl group, said alkyl group being
b) optionally substituted, especially with one or more identical or different radicals chosen from
   hydroxyl;
   (hetero)cycloalkyl, optionally substituted especially with one or more identical or different radicals chosen from hydroxyl, C₁-C₄ alkyl, C₁-C₄ hydroxyalkyl, C₁-C₄ alkoxy and/or at least one of the ring members of said (hetero)cycloalkyl denoting a ketone (—CO—);
   aryl, optionally substituted especially with one or more identical or different radicals chosen from C₁-C₄ alkyl, C₁-C₄ alkoxy, hydroxyl,

and/or said alkyl radical being
b) optionally interrupted with one or more heteroatoms such as O, S and N(R₅), with R₅ representing a hydrogen atom or a (C₁-C₄)alkyl group such as methyl;

ii) a (C₂-C₁)alkenyl group, said alkenyl radical being
b) optionally substituted, especially with one or more identical or different radicals chosen from
   hydroxyl,
   aryl, optionally substituted especially with one or more identical or different radicals chosen from C₁-C₄ alkyl, C₁-C₄ alkoxy, hydroxyl,
      and/or said alkenyl radical being
b) optionally interrupted with one or more heteroatoms such as O, S and N(R₅), with R₅ representing a hydrogen atom or a (C₁-C₄)alkyl group such as methyl;

iii) an optionally substituted aryl or heteroaryl radical;

Ta represents a radical chosen from:
i) a hydrogen atom;
ii) a (C₂-C₁₈)alkyl group, said alkyl group being
   b) optionally substituted, especially with one or more identical or different radicals chosen from
      hydroxyl,
      carboxy,
      (C₁-C₆)alkyloxycarbonyl,
      aryl, optionally substituted especially with one or more identical or different radicals chosen from C₁-C₄ alkyl, C₁-C₄ alkoxy, hydroxyl,
and/or said alkyl radical being
   b) optionally interrupted with one or more heteroatoms such as O, S and N(R₅), with R₅ representing a hydrogen atom or a (C₁-C₄)alkyl group such as methyl;
iii) a (C₂-C₁)alkenyl group, said alkenyl group being
   b) optionally substituted, especially with one or more identical or different radicals chosen from
      hydroxyl,
      carboxy,
      (C₁-C₆)alkyloxycarbonyl,
      aryl, optionally substituted especially with one or more identical or different radicals chosen from C₁-C₄ alkyl, C₁-C₄ alkoxy, hydroxyl,
and/or said alkenyl radical being
   b) optionally interrupted with one or more heteroatoms such as O, S and N(R₅), with R₅ representing a hydrogen atom or a (C₁-C₄)alkyl group such as methyl;
iv) an aryl or heteroaryl radical, optionally substituted especially with one or more identical or different radicals chosen from C₁-C₄ alkyl, hydroxyl, C₁-C₄ alkoxy, such as methoxy;
v) a heterocycloalkyl radical, optionally substituted especially with one or more identical or different radicals chosen from hydroxyl, C₁-C₄ hydroxyalkyl, C₁-C₄ alkyl, C₁-C₄ alkoxy and/or at least one of the ring members of said heterocycloalkyl radical denoting a ketone (—CO—);

Tb represents a radical chosen from:
i) a hydrogen atom;
ii) a (C₁-C₁)alkyl group, said alkyl group being
   c) optionally substituted, especially with one or more identical or different radicals chosen from
      hydroxyl,
      carboxy,
      (C₁-C₆)alkyloxycarbonyl,
      aryl, optionally substituted especially with one or more identical or different radicals chosen from C₁-C₄ alkyl, C₁-C₄ alkoxy, hydroxyl,
and/or said alkyl radical being
   b) optionally interrupted with one or more heteroatoms such as O, S and N(R₅), with R₅ representing a hydrogen atom or a (C₁-C₄)alkyl group such as methyl;
iii) a (C₂-C₁)alkenyl group, said alkenyl group being
   c) optionally substituted, especially with one or more identical or different radicals chosen from
      hydroxyl,
      carboxy,
      (C₁-C₆)alkyloxycarbonyl,
      aryl, optionally substituted especially with one or more identical or different radicals chosen from C₁-C₄ alkyl, C₁-C₄ alkoxy, hydroxyl,
and/or said alkenyl radical being
   b) optionally interrupted with one or more heteroatoms such as O, S and N(R₅), with R₅ representing a hydrogen atom or a (C₁-C₄)alkyl group such as methyl;
iv) an aryl or heteroaryl radical, optionally substituted especially with one or more identical or different radicals chosen from C₁-C₄ alkyl, hydroxyl, C₁-C₄ alkoxy, such as methoxy;
v) a heterocycloalkyl radical, optionally substituted especially with one or more identical or different radicals chosen from hydroxyl, C₁-C₄ hydroxyalkyl, C₁-C₄ alkyl, C₁-C₄ alkoxy and/or at least one of the ring members of said heterocycloalkyl radical denoting a ketone (—CO—);
   or else Ta and Tb form, together with the nitrogen atom to which they are attached, an optionally substituted heterocycloalkyl, such as piperazino, piperidino or morpholino;
   and also the salts thereof, the solvates thereof and the optical and/or geometrical isomers thereof, including enantiomers and diastereoisomers, and the racemates thereof, alone or in a mixture.

As examples of novel compounds of formula (I) and of formula (F2), mention may be made of the following compounds 5′ to 15′ and 18′ to 20′:

| | | Com- |
|---|---|---|
| Structure | Chemical name | pound |
| 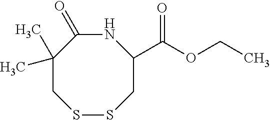 [SMILES: CCOC(=O)C1CSSCC(C)(C)C(=O)N1] | ethyl 7,7-dimethyl-6- oxo-1,2,5- dithiazocane-4- carboxylate | 5′ |
| 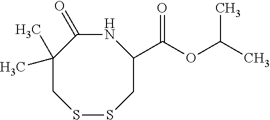 [SMILES: CC(C)OC(=O)C1CSSCC(C)(C)C(=O)N1] | propan-2-yl 7,7- dimethyl-6-oxo-1,2,5- dithiazocane-4- carboxylate | 6′ |
| 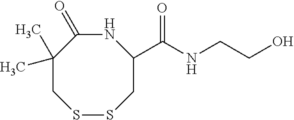 [SMILES: CC1(C)CSSCC(C(=O)NCCO)NC1=O] | N-(2-hydroxyethyl)- 7,7-dimethyl-6-oxo- 1,2,5-dithiazocane-4- carboxamide | 7′ |
| 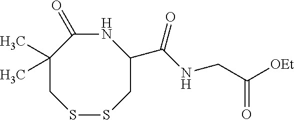 [SMILES: CCOC(=O)CNC(=O)C1CSSCC(C)(C)C(=O)N1] | ethyl N-[(7,7- dimethyl-6-oxo-1,2,5- dithiazocan-4- yl)carbonyl]glycinate | 8′ |
| 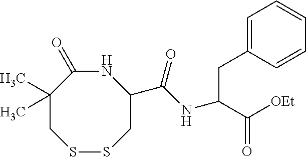 [SMILES: CCOC(=O)C(Cc1ccccc1)NC(=O)C1CSSCC(C)(C)C(=O)N1] | ethyl N-[(7,7- dimethyl-6-oxo-1,2,5- dithiazocan-4- yl)carbonyl]phenyl- alaninate | 9′ |
| 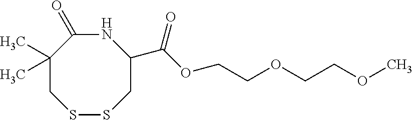 | 2-(2- methoxyethoxy)ethyl 7,7-dimethyl-6-oxo- 1,2,5-dithiazocane-4- carboxylate | 10′ |
| 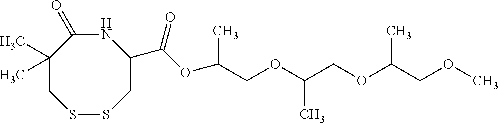 | 1-({1-[(1- methoxypropan-2- yl)oxy]propan-2- yl}oxy)propan-2-yl 7,7-dimethyl-6-oxo- 1,2,5-dithiazocane-4- carboxylate | 11′ |
| 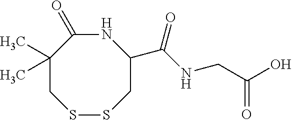 [SMILES: CC1(C)CSSCC(C(=O)NCC(=O)O)NC1=O] | N-[(7,7-dimethyl-6- oxo-1,2,5- dithiazocan-4- yl)carbonyl]glycine | 12′ |
| 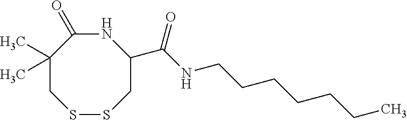 [SMILES: CCCCCCCNC(=O)C1CSSCC(C)(C)C(=O)N1] | N-heptyl-7,7- dimethyl-6-oxo-1,2,5- dithiazocane-4- carboxamide | 13′ |
| 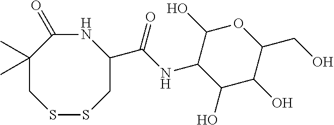 [SMILES: CC1(C)CSSCC(C(=O)NC2C(O)OC(CO)C(O)C2O)NC1=O] | 2-deoxy-2-{[(7,7- dimethyl-6-oxo-1,2,5- dithiazocan-4- yl)carbonyl]amino}hexo- pyranose | 14′ |
| 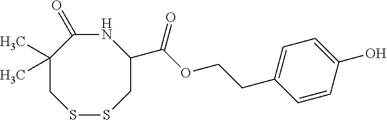 [SMILES: CC1(C)CSSCC(C(=O)OCCc2ccc(O)cc2)NC1=O] | 2-(4- hydroxyphenyl)ethyl 7,7-dimethyl-6-oxo- 1,2,5-dithiazocane-4- carboxylate | 15′ |
| 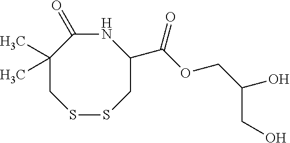 [SMILES: CC1(C)CSSCC(C(=O)OCC(O)CO)NC1=O] | 2,3-dihydroxypropyl 7,7-dimethyl-6-oxo- 1,2,5-dithiazocane-4- carboxylate | 18′ |
| 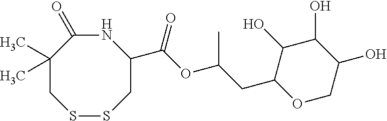 [SMILES: CC(CC1OCC(O)C(O)C1O)OC(=O)C1CSSCC(C)(C)C(=O)N1] | 4,8-anhydro-1,3- dideoxy-2-O-[(7,7- dimethyl-6-oxo-1,2,5- dithiazocan-4- yl)carbonyl]octitol | 19′ |
| 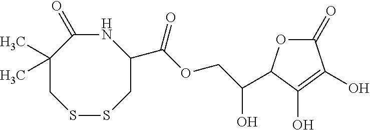 [SMILES: CC1(C)CSSCC(C(=O)OCC(O)C2OC(=O)C(O)=C2O)NC1=O] | 2-(3,4-dihydroxy-5- oxo-2,5-dihydrofuran- 2-yl)-2-hydroxyethyl 7,7-dimethyl-6-oxo- 1,2,5-dithiazocane-4- carboxylate | 20′ |

and also the salts thereof, the solvates thereof and including enantiomers and diastereoisomers, and the racemates thereof, alone or in a mixture, and in particular the R isomers of the carbon in position 4 of the dithiazocane ring of said compounds.

TABLE 1
The numbers in the table correspond to the numbers used in the
examples below:

| Com- | | |
|---|---|---|
| pound | | |
| No. | Structure | Chemical name |
| 1 | 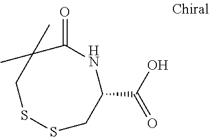 | 7,7-dimethyl-6-oxo-1,2,5- dithiazocane-4-(R)- carboxylic acid |
| 2 | 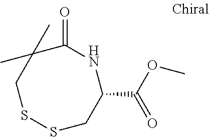 | methyl 7,7-dimethyl-6-oxo- 1,2,5-dithiazocane-4(R)- carboxylate |

and also the salts thereof, the solvates thereof and including enantiomers and diastereoisomers, and the racemates thereof, alone or in a mixture.

The compounds of the invention may be prepared according to the following scheme 1, making use of the methods known and described for those skilled in the art in Synthesis and pharmacological activities of novel cyclic disulfide and cyclic sulfide derivatives as hepatoprotective agents, by Ito, Susumu et al., from Chemical & Pharmaceutical Bulletin, 41(6), 1066-73; 1993 and in patent EP 356006A1.

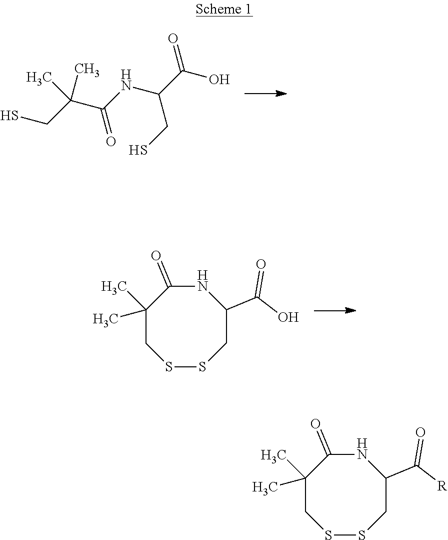

Generally, after obtaining the ring having a carboxylic acid function, the latter may be converted to ester or amide according to reactions known to those skilled in the art and described in: Comprehensive Organic Transformations, second edition, by RC Larock, ISBN 978-1-118-03758-4, 2010 Wiley.

### Composition

The compounds of formula (I) according to the invention have a most particular application in the cosmetics field.

Another subject of the invention is a composition, preferably a cosmetic composition, containing at least one compound of formula (I), in particular a composition containing at least one compound chosen from the compounds 1′ to 15′ and 18′ to 20′ described previously, and more preferentially containing at least one compound chosen from the compounds 1 and 2 described previously, most particularly containing the compound 1.

Another subject of the invention is a composition, especially a cosmetic composition, containing at least one novel compound of formula (F1) or of formula (F2) as described previously, in particular containing at least one novel compound of formula (F2), more particularly containing at least one compound of formula 5′ to 15′ and 18′ to 20′ as defined previously, said composition containing in particular at least one novel compound of formula 5′ to 15′ and 18′ to 20′ in R isomer form (isomerism of the carbon bearing the radical CO-Sub′ in the novel compounds of formula (F2)).

The composition, preferably cosmetic composition, according to the invention is advantageously a composition intended for topical application.

In a variant of the invention, the composition may be a dermatological or pharmaceutical composition, especially intended for topical application.

The composition, preferably cosmetic composition, according to the invention advantageously comprises, in a physiologically acceptable medium, at least one compound of formula (I) as described previously, in particular at least one compound chosen from the compounds 1′ to 15′ and 18′ to 20′ described previously, preferably at least one compound chosen from the compounds 1 or 2 described previously and most particularly the compound 1 described previously.

The term “physiologically acceptable medium” is intended to mean a medium that is compatible with human keratin materials such as the skin of the body, such as the hands, neck or face.

The compound of formula (I) may be present in the composition, preferably cosmetic composition, according to the invention at an amount that may be between 0.01% and 10% by weight, preferably between 0.1% and 5% by weight, especially from 0.5% to 3% by weight relative to the total weight of the composition.

The composition, preferably cosmetic composition, according to the invention may comprise water and/or adjuvants usually used in the cosmetics field.

Mention may be made especially of organic solvents, especially C2-C6 alcohols; oils, especially hydrocarbon-based oils and silicone oils; waxes, pigments, fillers, dyes, surfactants, emulsifiers; cosmetic active agents, organic or mineral photoprotective agents, polymers, thickeners, preserving agents, fragrances, bactericides, ceramides, odor absorbers, antioxidants.

These optional cosmetic adjuvants may be present in the cosmetic composition in a proportion of from 0.001% to 80% by weight and especially from 0.1% to 40% by weight relative to the total weight of the composition. In any case, these adjuvants, and the proportions thereof, will be chosen by those skilled in the art such that the advantageous properties of the compounds according to the invention are not, or are not substantially, adversely affected by the envisaged addition.

As active agents, it will be advantageous to introduce into the composition according to the invention at least one compound chosen from: desquamating agents; calmatives, organic or mineral photoprotective agents, moisturizers; additional depigmenting agents other than the compounds of formula (I) of the invention; anti-glycation agents; NO-synthase inhibitors; agents for stimulating the synthesis of dermal or epidermal macromolecules and/or for preventing their degradation; agents for stimulating fibroblast and/or keratinocyte proliferation or for stimulating keratinocyte differentiation; dermo-decontracting agents; tensioning agents; anti-pollution agents and/or free-radical scavengers; agents acting on the microcirculation; agents acting on the energy metabolism of cells; and mixtures thereof.

The composition, preferably cosmetic composition, according to the invention may in particular be in any presentation form normally used in the cosmetics field, and especially in the form of an aqueous or aqueous-alcoholic solution, which is optionally gelled, a dispersion of the lotion type, which is optionally a two-phase lotion, an oil-in-water or water-in-oil or multiple (for example W/O/W or O/W/O) emulsion, an aqueous gel, a dispersion of oil in an aqueous phase by means of spherules, these spherules possibly being polymer nanoparticles such as nanospheres and nanocapsules or, better still, lipid vesicles of the ionic and/or nonionic type; aqueous or oily gels. These compositions are prepared according to the usual methods. The cosmetic composition according to the invention may constitute a skincare composition, and especially a cleansing, protecting, treating or care cream for the face, the hands, the feet, the major anatomical folds or the body (for example day creams, night creams, makeup-removing creams, foundation creams or anti-sun creams); a fluid foundation, a makeup-removing milk, a protective or care body milk or an anti-sun milk; a skincare lotion, gel or foam, such as a cleansing lotion.

Another subject of the invention is a non-therapeutic cosmetic process for depigmenting, lightening and/or bleaching keratin materials, especially the skin, comprising the application of the composition, preferably cosmetic composition, previously described.

More preferably, it is a non-therapeutic cosmetic process for depigmenting, lightening and/or bleaching the skin.

The invention is illustrated in greater detail by the following nonlimiting examples.

The various examples below describe various synthesis routes making it possible to obtain the compounds of formula (I).

In these schemes, “rt” means room temperature.

### EXAMPLES 1 AND 2: SYNTHESIS OF COMPOUNDS 1 AND 2

By Ito, Susumu et al., from Chemical & Pharmaceutical Bulletin, 41(6), 1066-73; 1993 and in patent EP 356006A1 Procedure:

To a solution of N-(3-mercapto-2,2-dimethylpropionyl)-L-cysteine (31 g, 130 mmol) in 440 ml of ethyl acetate, the following are added: a solution of iodine (33 g, 130 mmol) in 440 ml of EtOAc, then dropwise, between −10 and +5° C., a solution of triethylamine (29 g, 290 mmol) in 430 ml of EtOAc over 3 h. The mixture is stirred at this temperature for 1 hour. The triethylammonium iodide precipitate formed is then filtered, and the filtrate is washed with 1 N hydrochloric acid, sodium bisulfite at 1% in water then a saturated sodium chloride solution. After concentrating the filtrate under vacuum, a precipitate forms which, after filtration, is recrystallized in a (1/5) ethanol/water mixture to give compound 1 (19.4 g, 63%).

The MS and NMR spectra are in accordance with the desired product.

Diazomethane dissolved in ether at 0° C. is added to a solution of compound 1 (11 g) acidified with hydrochloric acid (3.9 N in EtOAc) in methyl acetate (250 ml). At the end of the addition, the reaction mixture is stirred for 10 minutes before adding 8 ml of acetic acid thereto. After concentration under vacuum, the crude product is purified by silica column to give compound 2 with a yield of 88%.

The MS and NMR spectra are in accordance with the desired product.

### EXAMPLE 3: SYNTHESIS OF COMPOUND 3′

To a solution of compound 1 (1 eq, 500 mg) in anhydrous Tetrahydrofurane (THF) 5 ml at 0° C., we added oxalyle chloride (COCl)₂ (1.5 eq, 0.274 ml) and 2 drops of N,N-Dimethylformamide (DMF). The mixture was stirred for 4 hours and concentrated with toluene. The residue was solubilized in a mix of THF/dioxane/DMF (60 ml/30 ml/15 ml) and cool at 0° C. bubbling with ammonia during 1 hour and the stirring is continued for 16 hours at room temperature. The mixture was concentrated under reduced pressure and the residue was purified by chromatography on inverse silica gel (H₂O/Acetonitrile: 95/5 to 5/95) and lyophilized to give compound 3′ with a yield of 42%.

The MS and NMR spectra are in accordance with the desired product.

### EXAMPLE 4: SYNTHESIS OF COMPOUND 4′

To a solution of compound 1 (1 eq; 200 mg) in anhydrous N,N-Dimethylformamide (DMF) 4 ml, we added Me₂NH (2 eq, 2M/THF; 0.85 ml), 1-[Bis(dimethylamino)methylene]-1H-1,2,3-triazolo[4,5-b]pyridinium 3-oxid hexafluorophosphate (HATU) (1.1 eq; 355 mg) and N, N-diisopropyléthylamine (DIEA) (3 eq, 0.421 ml). The mixture was stirred for 20 hours at room temperature. Water was added to the mixture and extracted with Dicloromethane (DCM) (1 time), ethyl acetate (AcOEt) (1 time) and dichloromethane (DCM) (1 time). The organic layer was mixed and dried with Na₂SO₄, filtered and concentrated under reduced pressure. The residue obtained is purified by chromatography on silica gel (DCM/MeOH: 97/3) to give compound 4′, with a yield of 49%.

The MS and NMR spectra are in accordance with the desired product.

### EXAMPLE 5: SYNTHESIS OF COMPOUND 5′

To a solution of compound 1 (1 eq; 300 mg) in dichloromethane (DCM) 6 ml, we added successively N,N′-dicyclohexylcarbodiimide (DCC) (1.5 eq, 395 mg), Ethanol (5 eq, 0.37 ml) and 4-dimethylaminopyridine (DMAP) (0.1 eq, 15.6 mg). The mixture was stirred for 16 hours at room temperature. Brine was added to the mixture and extracted with ethyl acetate (AcOEt) (3 times). The organic layer was mixed and dried with Na₂SO₄, filtered and concentrated under reduced pressure. The residue obtained is purified by chromatography on silica gel (DCM/cyclohexane: 9/1) to give compound 5′ with a yield of 71%.

The MS and NMR spectra are in accordance with the desired product

### EXAMPLE 6: SYNTHESIS OF COMPOUND 6′

To a solution of compound 1 (1 eq, 250 mg) in dichloromethane (DCM) 5 ml, we added successively N,N′-dicyclohexylcarbodiimide (DCC) (1.5 eq, 328 mg), Isopropanol (5 eq, 0.41 ml) and 4-dimethylaminopyridine (DMAP) (0.1 eq, 13 mg). The mixture was stirred for 16 hours at room temperature. Brine was added to the mixture and extracted with ethyl acetate (AcOEt) (3 times). The organic layer was mixed and dried with Na₂SO₄, filtered and concentrated under reduced pressure. The residue obtained is purified by chromatography on silica gel (cyclohexane/AcOEt: 8/2) to give compound 6′, with a yield of 72%.

The MS and NMR spectra are in accordance with the desired product

### EXAMPLE 7: SYNTHESIS OF COMPOUND 7′

To a solution of compound 1 (1 eq, 400 mg) in anhydrous N,N-Dimethylformamide (DMF) 8 ml, we added (2-aminoethoxy)(tert-butyl)dimethylsilane (2 eq, 596 mg), 1-[Bis(dimethylamino)methylene]-1H-1,2,3-triazolo[4,5-b]pyridinium 3-oxid hexafluorophosphate (HATU) (1.1 eq, 732 mg) and N, N-diisopropyléthylamine (DIEA) (3 eq, 0.843 ml). The mixture was stirred for 40 hours at room temperature. Water was added to the mixture and extracted with dichloromethane (DCM) (1 time), AcOEt (1 time) and dichloromethane (DCM) (1 time). The organic layer was mixed and dried with Na₂SO₄, filtered and concentrated under reduced pressure. The residue obtained is purified by chromatography on silica gel (DCM/acetone 99/1 to 95/5) to give intermediate compound with a yield of 55%.

The compound obtained (1 eq, 370 mg) previously was dissolved in anhydrous DCM 10 ml, HCl (4N/dioxane, 2 eq, 0.47 ml) was added at 0° C. The mixture was stirred for 20 hours at 0° C. after it was concentrated under reduced pressure. The residue is purified by chromatography on silica gel (3 times DCM/EtOH: 95/5 to 92/8 and 1 time AcOEt/MeOH: 97/3) to give compound 7′ with a yield of 27%.

The MS and NMR spectra are in accordance with the desired product

### EXAMPLE 8: SYNTHESIS OF COMPOUND 8′

To a solution of compound 1 (1 eq, 400 mg) in anhydrous N,N-Dimethylformamide (DMF)₁₂ ml, we added at 0° C. under Argon, ethyl-2-(chloroamino)-acetate (2 eq, 303 mg), 1-[Bis(dimethylamino)methylene]-1H-1,2,3-triazolo[4,5-b]pyridinium 3-oxid hexafluorophosphate (HATU) (1.1 eq, 732 mg) and N, N-diisopropyléthylamine (DIEA) (5 eq, 1.38 ml). The mixture was stirred for 40 hours at room temperature after it was concentrated under reduced pressure. The residue obtained is purified by chromatography on silica gel (DCM/AcOEt: 85/15 to 75/15 (2 times)) to give compound 8′ with a yield of 70%.

The MS and NMR spectra are in accordance with the desired product.

### EXAMPLE 9: SYNTHESIS OF COMPOUND 9′

To a solution of compound 1 (1 eq; 150 mg) in anhydrous N,N-Dimethylformamide (DMF) 5 ml, we added at 0° C. under Argon, ethyl-2-(chloroamino)-3-phenyl-propanoate (1.3 eq, 187 mg), 1-[Bis(dimethylamino)methylene]-1H-1,2,3-triazolo[4,5-b]pyridinium 3-oxid hexafluorophosphate (HATU) (1.1 eq, 286 mg) and DIEA (5 eq, 0.55 ml). The mixture was stirred for 40 hours at room temperature after it was concentrated under reduced pressure. The residue obtained is purified by chromatography on silica gel (DCM/AcOEt: 85/15 to 8/2) to give compound 9′ with a yield of 89%.

The MS and NMR spectra are in accordance with the desired product

### EXAMPLE 10: SYNTHESIS OF COMPOUND 10′

To a solution of compound 1 (1 eq, 250 mg) in dichloromethane (DCM) 5 ml, we added successively N,N′-dicyclohexylcarbodiimide (DCC) (1.5 eq, 328 mg), 2-(2-methoxyethoxy)ethanol (5 eq, 0.632 ml) and 4-dimethylaminopyridine (DMAP) (0.2 eq, 26 mg). The mixture was stirred for 16 hours at room temperature. Brine was added to the mixture and extracted with ethyl acetate (AcOEt) (3 times). The organic layer was mixed and dried with Na₂SO₄, filtered and concentrated under reduced pressure. The residue obtained is purified by chromatography on silica gel (DCM/AcOEt: 85/15 (6 times)) to give compound 10′ with a yield of 52%.

The MS and NMR spectra are in accordance with the desired product.

### EXAMPLE 11: SYNTHESIS OF COMPOUND 11′

To a solution of compound 1 (1 eq, 250 mg) in dichloromethane (DCM) 5 ml, we added successively N,N′-dicyclohexylcarbodiimide (DCC) (1.5 eq, 328 mg), 1-({1-[(1-methoxypropan-2-yl}oxy]propan-2-yl)oxy)popan-2-ol (5 eq, 1.15 g) and 4-dimethylaminopyridine (DMAP) (0.2 eq, 26 mg). The mixture was stirred for 16 hours at room temperature. Brine was added to the mixture and extracted with ethyl acetate (AcOEt) (3 times). The organic layer was mixed and dried with Na₂SO₄, filtered and concentrated under reduced pressure. The residue obtained is purified by chromatography on silica gel (DCM/AcOEt: 85/15 (5 times)) to give compound 11′ with a yield of 54%.

The MS and NMR spectra are in accordance with the desired product.

### EXAMPLE 12: SYNTHESIS OF COMPOUND 12′

To a solution of compound 1 (1 eq, 400 mg) with N-hydroxysuccinimide (1.5 eq, 293 mg) in N,N-Dimethylformamide (DMF) 4 ml at 0° C., we added slowly N,N′-dicyclohexylcarbodiimide (DCC) (1.5 eq, 526 mg) in solution in N,N-Dimethylformamide (DMF) 3 ml. The mixture was stirred for 2 hours at room temperature and a solution of glycine (1.1 eq, 140 mg) with KHCO₃ (2 eq, 340 mg) in water (10 ml) was added. The mixture was stirred for 16 hours at room temperature and cool with ice bath. Water (5 ml) was added and slowly formic acid was added pH 3.

The mixture was lyophilized. The crude was purified by chromatography on inverse silica gel (H₂O/Acetonitrile: 95/5 to 5/95) to give compound 12′ with a yield of 36%.

The MS and NMR spectra are in accordance with the desired product.

### EXAMPLE 13: SYNTHESIS OF COMPOUND 13′

To a solution of compound 1 (1 eq; 300 mg) in anhydrous N,N-Dimethylformamide (DMF) 6 ml, we added reactant heptylamine (2 eq, 0.838 ml), 1-[Bis(dimethylamino)methylene]-1H-1,2,3-triazolo[4,5-b]pyridinium 3-oxid hexafluorophosphate (HATU) (1.1 eq; 549 mg) and N, N-diisopropyléthylamine (DIEA) (3 eq, 0.632 ml). The mixture was stirred for 40 hours at room temperature. Water was added to the mixture and extracted with Dicloromethane (DCM) (1 time), ethyl acetate (AcOEt) (1 time) and dichloromethane (DCM) (1 time). The organic layer was mixed and dried with Na₂SO₄, filtered and concentrated under reduced pressure. The residue obtained is purified by chromatography on silica gel (DCM/Acetone: 95/5 (2 times) and DCM/AcOEt: 8/2 (2 times)) to give compound 13′ with a yield of 48%.

The MS and NMR spectra are in accordance with the desired product.

### EXAMPLE 14: SYNTHESIS OF COMPOUND 14′

To a suspension of D-(+)-glucosamine.HCl (1 eq, 400 mg) in pyridine (8 ml), we added successively hexamethyldisilazane (HMDS) (10 eq, 3.94 ml) and chlorotrimethylsilane (10 eq, 2.37 ml). The mixture was stirred for 20 hours at room temperature after it was concentrated under reduced pressure with toluene and purified by chromatography on silica gel (cyclohexane/AcOEt: 9/1). A colorless oil is obtained, yield 63%

To a solution of compound 1 (1 eq, 178 mg) in N,N-Dimethylformamide (DMF) 4.5 ml, we added successively the compound synthesized previously (1.1 eq, 390 mg), 1-[Bis(dimethylamino)methylene]-1H-1,2,3-triazolo[4,5-b]pyridinium 3-oxid hexafluorophosphate (HATU) (1.2 eq, 345 mg) and N, N-diisopropyléthylamine (DIEA) (5 eq, 0.63 ml). The mixture was stirred for 20 hours at room temperature. Water (30 ml) was added and the solution was lyophilized. The crude was used for the next step.

The crude was solubilized in a mix of DCM/MeOH 8/2 (15 ml) after silicagel and a little of AcOH (0.1 ml) was added. The mixture was stirred for 2 days at room temperature before to be filtered and concentrated under reduced pressure. The crude was purified by chromatography on inverse silica gel (H₂O/ACN: 98/2 to 95/5). We obtained a white powder with global yield of 77% for the three steps.

The MS and NMR spectra are in accordance with the desired product.

### EXAMPLE 15: SYNTHESIS OF COMPOUND 18′

To a solution of compound 1 (1 eq, 400 mg) in dichloromethane (DCM)/N,N-Dimethylformamide (DMF) (6 ml/3 ml), we added N,N′-dicyclohexylcarbodiimide (DCC) (1.5 eq, 526 mg). The mixture was stirred for 5 min at room temperature after solketal (5 eq, 1.09 ml) and 4-dimethylaminopyridine (DMAP) (0.1 eq, 20 mg) was added. The mixture was stirred for 16 hours at room temperature. Dicloromethane (DCM) was added and the mixture was filtered on Büchner. The solution obtained was concentrated under reduced pressure and purified by chromatography on silica gel (DCM/acetone: 95/5). A white powder is obtained, yield 23%.

A solution of the compound synthesized previously (1 eq, 258 mg) in AcOH (8 ml) with water (1.95 ml) was stirred for 2 days at room temperature and lyophilized. A white hygroscopic powder (quantitative yield) was obtained. The MS and NMR spectra are in accordance with the desired product.

### EXAMPLE 16: SYNTHESIS OF COMPOUND 19′

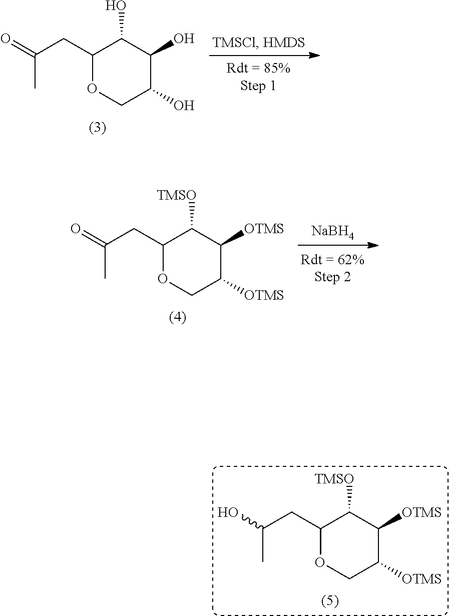

To suspension of compound (3) above (1 eq, 2 g) in pyridine (45.1 ml), we added successively hexamethyldisilazane (HMDS (1 eq 223 ml) and chlorotrimethylsilane (10 eq, 13.4 ml). The mixture was stirred for 16 hours at room temperature after it was concentrated under reduced pressure with toluene and purified by chromatography on silica gel (cyclohexane/AcOEt: 9/1). A pale yellow oil is obtained, yield 85%.

To a solution of previous compound (4) above (1 eq, 1 g) in EtOH (30 ml), we added NaBH₄ (1 eq, 0.093 g). The mixture was stirred for 1 hour at room temperature. The mixture is then poured in saturated aqueous solution of NH₄Cl, filtered and concentrated under reduced pressure. The residue was purified by chromatography on silica gel (cyclohexane/AcOEt: 8/2). We obtained a colorless oil compound (5) with yield 62%.

To a solution of compound 1 (1 eq, 200 mg) in anhydrous tétrahydrofurane (THF) 16 ml we added 1,1′-Carbonyldiimidazole (CDI) (1.2 eq, 165 mg), and the mixture was stirred at room temperature for 3 h. Compound (5) below (1.2 eq, 416 mg) in solution in anhydrous THF (3 ml) was added and the mixture was heated at 60° C. during 2 days.

After cool, the mixture was diluted in a solution of DCM/MeOH 8/2 (20 ml), and a little of AcOH (1 ml) was added. The solution was stirred for 2 days and concentrated under reduced pressure with toluene. The oil obtained was purified by chromatography on inverse silica gel (H₂O/ACN: 98/2 to 5/95). We obtained a white powder with yield 19%.

The MS and NMR spectra are in accordance with the desired product

### EXAMPLE 17: DEMONSTRATION OF THE DEPIGMENTING ACTIVITY

The measurement of the depigmenting activity (reduction of melanin production) of compounds of formula (I) was performed by assaying normal human melanocytes in vitro as follows.

First of all, normal human melanocytes are cultured and dispensed into wells. After 24 hours, the culture medium was replaced with a medium containing compounds of formula (I) to be evaluated. The cells were incubated 72 hours before measurement of the final optical density, which measures the amount of melanin produced by the melanocytes. A dose effect is performed using a wide concentration range of the compounds evaluated. Thus, by making the concentrations and the melanin measurements correspond, it is possible to determine an IC50 in μM: concentration at which a 50% decrease in melanin synthesis is achieved.

Various test campaigns were conducted and the results collated in the table below.

| | | | Maximum |
|---|---|---|---|
| | | | concentration |
| Compound | | IC50 | on tested |
| No. | Structure | (μM) | (μM) |
| Compound 1 | 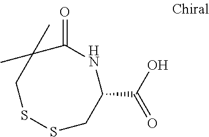 | 62.5 | 200 |
| Compound 2 | 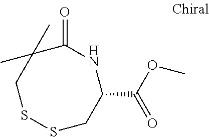 | 13.6 | 200 |
| Compound 3′ | 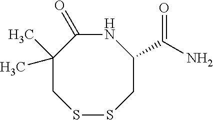 | 2.35 | 200 |
| Compound 4′ | 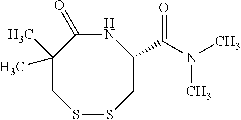 | 4.23 | 200 |
| Compound 5′ | 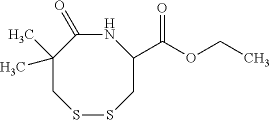 [SMILES: CCOC(=O)C1CSSCC(C)(C)C(=O)N1] | 1, 12 | 200 |
| Compound 6′ | 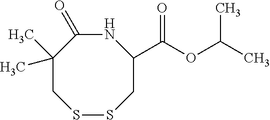 [SMILES: CC(C)OC(=O)C1CSSCC(C)(C)C(=O)N1] | 1.9 | 200 |
| Compound 7′ | 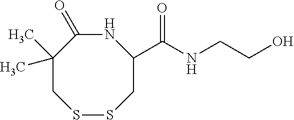 [SMILES: CC1(C)CSSCC(C(=O)NCCO)NC1=O] | <0.4 | 200 |
| Compound 8′ | 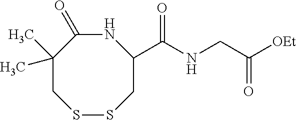 [SMILES: CCOC(=O)CNC(=O)C1CSSCC(C)(C)C(=O)N1] | 0.87 | 200 |
| Compound 9′ | 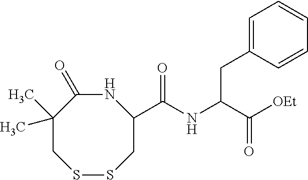 [SMILES: CCOC(=O)C(Cc1ccccc1)NC(=O)C1CSSCC(C)(C)C(=O)N1] | 20.8 | 200 |
| Compound 10′ | 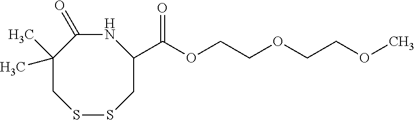 | 5.26 | 200 |
| Compound 11′ | 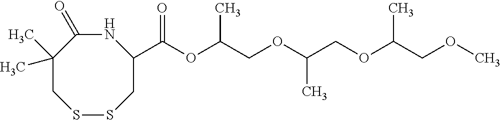 | 10.7 | 200 |
| Compound 12′ | 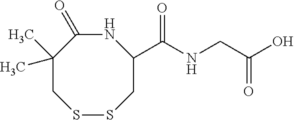 [SMILES: CC1(C)CSSCC(C(=O)NCC(=O)O)NC1=O] | 63.8 | 200 |
| Compound 13′ | 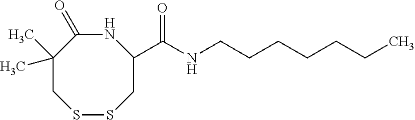 [SMILES: CCCCCCCNC(=O)C1CSSCC(C)(C)C(=O)N1] | 0.66 | 200 |
| Compound 18′ | 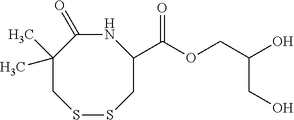 [SMILES: CC1(C)CSSCC(C(=O)OCC(O)CO)NC1=O] | 9.8 | 200 |

The compounds of formula (I) showed a strong depigmenting effect.

Coculture Experiment:

A biological test demonstrated the depigmenting activity of compound 1.

The melanogenesis-modulating effect of compound 1 was measured according to the method described in patent FR-A-2734825, and also in the article by R. Schmidt, P. Krien and M. Régnier, Anal. Biochem., 235(2), 113-18, 1996. This test is performed on a coculture of keratinocytes and melanocytes.

The following were determined for the test compound:
the cytotoxicity,
the inhibitory activity on melanin synthesis, by estimating the melanin optical density.

The IC50 values (concentration for which 50% of the melanin synthesis is inhibited) were determined.

Compound 1: non-cytotoxic, IC50=9.6 μM.
Compound 2: non-cytotoxic IC50=0.74 μM.
Compound 5: non-cytotoxic IC50=0.79 μM.
Compound 6: non-cytotoxic IC50=1.57 μM.
Compound 7: non-cytotoxic IC50=1.04 μM.
Compound 8: non-cytotoxic IC50=1.23 μM.
Compound 10: non-cytotoxic IC50=7.62 μM.

### EXAMPLE 18: OTHER DEMONSTRATION OF THE DEPIGMENTING ACTIVITY

Compositions comprising 300 μM of compound 1 in DMSO were applied to samples of pigmented reconstructed epidermis (cf. EP 1 878 790). The control is DMSO.

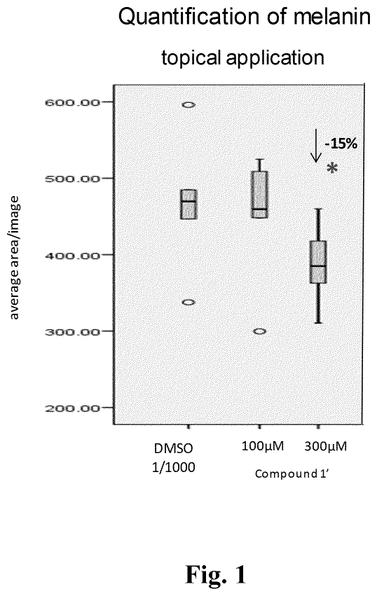

The melanin was quantified by image analysis on histological slices after staining with Fontana Masson dye. Each sample of coloured epidermis is photographed over its entire length using a camera connected to a microscope. The melanin is thresholded and the number of melanin pixels is measured in each field using automated image analysis software. A non-parametric statistical test is performed in order to determine the significance of the measurements (Mann-Whitney test, see FIG. 1).

The pigmented reconstructed epidermis standard study model was published by: Regnier M, Duval C, Galey J B, Philippe M, Lagrange A, Tuloup R, Schmidt R, Cellular and Molecular Biology, 1999, 45, 7, 969-980: “Keratinocyte-Melanocyte co-cultures and pigmented reconstructed human epidermis: models to study modulation of melanogenesis”.

Significant depigmenting activity was evaluated at 300 μM for compound 1 (pValue<0.05: significant depigmenting activity).

### EXAMPLE 19: COSMETIC COMPOSITION

A skin depigmenting composition is prepared, comprising (in grams):

| | |
|---|---|
| Compound No. 1 | 2 g |
| PEG400 | 68 g |
| Ethanol | 30 g |

The composition applied to the skin makes it possible to lighten the skin of the face and especially to attenuate brown blemishes.

### EXAMPLE 20: GEL

A skin depigmenting gel is prepared, comprising (% by weight):

| | |
|---|---|
| Compound No. 1 | 0.25% |
| Carbomer (Carbopol 981 from Lubrizol) | 1% |
| Preservative | qs |
| Water | qs 100% |

The composition applied to the skin makes it possible to attenuate brown blemishes.

1.-17. (canceled)

18. A method for bleaching, lightening, and/or depigmenting a keratin material, comprising applying to the keratin material a composition comprising at least one compound of formula (I), 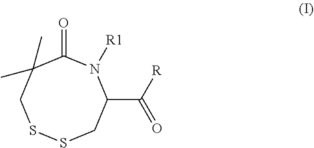
salts thereof, solvates thereof, optical and/or geometrical isomers thereof, including enantiomers and diastereoisomers thereof, racemates thereof, or mixtures thereof:
wherein in formula (I):
R is chosen from a radical OR′ or NRₐR_{b};
R₁ is chosen from a hydrogen atom or a (C₁-C₁₈)alkyl group;
R′ is chosen from:
i) a hydrogen atom;
ii) a (C₁-C₁₈)alkyl group, wherein:
a) the (C₁-C₁₈)alkyl group is optionally substituted with at least one identical or different radical chosen from:
hydroxyl;
(hetero)cycloalkyl, optionally substituted with at least one identical or different radical chosen from hydroxyl, C₁-C₄ alkyl, C₁-C₄ hydroxyalkyl, or C₁-C₄ alkoxy, and/or at least one of ring members of the (hetero)cycloalkyl denoting a ketone (—CO—); or
aryl, optionally substituted with at least one identical or different radical chosen from C₁-C₄ alkyl, C₁-C₄ alkoxy, or hydroxyl; and/or
b) the (C₁-C₁₈)alkyl group is optionally interrupted with at least one heteroatom;
iii) a (C₂-C₁₈)alkenyl group, wherein:
a) the (C₂-C₁₈)alkenyl group is optionally substituted with at least one identical or different radical chosen from:
hydroxyl; or
aryl, optionally substituted with at least one identical or different radical chosen from C₁-C₄ alkyl, C₁-C₄ alkoxy, or hydroxyl; and/or
b) the (C₂-C₁₈)alkenyl group is optionally interrupted with at least one heteroatom; or
iv) an optionally substituted aryl or heteroaryl radical;
Rₐ and R_{b} are independently chosen from:
i) a hydrogen atom;
ii) a (C₁-C₁₈)alkyl group, wherein:
a) the (C₁-C₁₈)alkyl group is optionally substituted with at least one identical or different radical chosen from:
hydroxyl;
carboxy;
(C₁-C₆)alkyloxycarbonyl; or
aryl, optionally substituted with at least one identical or different radical chosen from C₁-C₄ alkyl, C₁-C₄ alkoxy, or hydroxyl; and/or
b) the (C₁-C₁₈)alkyl group is optionally interrupted with at least one heteroatom;
iii) a (C₂-C₁₈)alkenyl group, wherein:
a) the (C₂-C₁₈)alkenyl group is optionally substituted with at least one identical or different radical chosen from:
hydroxyl;
carboxy;
(C₁-C₆)alkyloxycarbonyl; or
aryl; optionally substituted with at least one identical or different radical chosen from C₁-C₄ alkyl, C₁-C₄ alkoxy, or hydroxyl; and/or
b) the (C₂-C₁₈)alkenyl group is optionally interrupted with at least one heteroatom;
iv) an aryl or heteroaryl radical, optionally substituted with at least one identical or different radical chosen from C₁-C₄ alkyl, hydroxyl, or C₁-C₄ alkoxy; or
v) a heterocycloalkyl radical, optionally substituted with at least one identical or different radical chosen from hydroxyl, C₁-C₄ hydroxyalkyl, C₁-C₄ alkyl, or C₁-C₄ alkoxy, and/or at least one of ring members of the heterocycloalkyl radical denoting a ketone (—CO—); or
Rₐ and R_{b} form, together with the nitrogen atom to which Rₐ and R_{b} are attached, an optionally substituted heterocycloalkyl.

19. The method of claim 18, wherein the at least one compound of formula (I) is of R configuration.

20. The method of claim 18, wherein in formula (I), R₁ is chosen from a (C₁-C₆)alkyl group or a hydrogen atom.

21. The method of claim 18, wherein in formula (I), R is an OR′ radical, in which R′ is chosen from:
A) a hydrogen atom or a (C₁-C₄)alkyl group;
B) a (C₁-C₆) alkyl group optionally substituted with at least one identical or different radical chosen from:
hydroxyl;
heterocycloalkyl substituted with at least one identical or different radical chosen from hydroxyl, C₁-C₄ alkyl, C₁-C₄ hydroxyalkyl, or C₁-C₄ alkoxy, and/or one of the ring members of the heterocycloalkyl is a ketone (—CO—); or
phenyl, optionally substituted with at least one identical or different radical chosen from C₁-C₄ alkyl, C₁-C₄ alkoxy, or hydroxyl;
C) a (C₁-C₁₄)alkyl group, the (C₁-C₁₄) alkyl group being interrupted with at least one heteroatom;
D) a radical of formula (B′1): 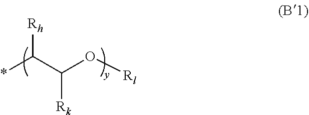
wherein Rₕ and Rₖ are independently chosen from a hydrogen atom or a methyl radical; Rₗ is H or OH; and y denotes an integer from 1 to 10; or
E) a (C₁-C₆) alkenyl group, optionally substituted with at least one identical or different radical chosen from:
hydroxyl;
heterocycloalkyl substituted with at least one identical or different radical chosen from hydroxyl, C₁-C₄ alkyl, C₁-C₄ hydroxyalkyl, or C₁-C₄ alkoxy; and/or one of the ring members of the heterocycloalkyl is a ketone (—CO—); or
phenyl, optionally substituted with at least one identical or different radical chosen from C₁-C₄ alkyl, C₁-C₄ alkoxy, or hydroxyl.

22. The method of claim 18, wherein in formula (I), R is an NRₐR_{b} radical, wherein:
A) Rₐ and R_{b} are independently chosen from a hydrogen atom or a (C₁-C₁₈)alkyl group;
B) Rₐ is a hydrogen atom and R_{b} is chosen from a (C₁-C₄)alkyl group, the (C₁-C₄)alkyl group being optionally substituted with at least one identical or different radical chosen from:
hydroxyl,
carboxy,
(C₁-C₆)alkyloxycarbonyl, or
phenyl, optionally substituted with at least one identical or different radical chosen from C₁-C₄ alkyl, C₁-C₄ alkoxy, or hydroxyl; or
C) Rₐ is a hydrogen atom and R_{b} represents a radical of the following formula B′2: 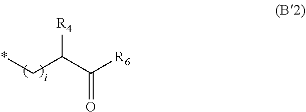
wherein:
i=0 or 1;
R₄ is chosen from a hydrogen atom or a (C₁-C₆)alkyl group optionally substituted with at least one group chosen from:
i) —Z—C(Z′)—Z″—R_{C}, wherein Z, Z′, and Z″, which are identical or different, represent an oxygen atom, a sulfur atom, or N(R_{D}), wherein R_{C} and R_{D}, which are identical or different, are chosen from a hydrogen atom or a (C₁-C₄)alkyl group;
ii) (hetero)aryl, optionally substituted with a hydroxyl group;
iii) (di)(C₁-C₄)(alkyl)amino; iv) —C(Z′)—Z″—R_{C}, wherein Z′, Z″ and R_{C} are defined as above; or v) —Z′″—R_{C}, wherein Z′″ is chosen from an oxygen, a sulfur atom, a selenium atom, or an NH group, and R_{C} is as defined previously;
or, when i=0, Rₐ and R₄ form, together with the nitrogen atom which bears Rₐ and with the carbon atom which bears R₄, a saturated 5- or 6-membered heterocycle;
R₆ is chosen from:
i) a hydroxyl radical —OH;
ii) a saturated or unsaturated (C₁-C₆)alkoxy radical; or
iii) an —NRfRg radical, wherein Rf and Rg, which are identical or different, are chosen from a hydrogen atom or a (C₁-C₆)alkyl group;
D) Rₐ is a hydrogen atom and R_{b} represents a radical of formula (B′2) as defined above, wherein in formula (B′2), R₆ is a hydroxyl radical or a saturated or unsaturated (C₁-C₆)alkoxy radical, and R₄ is a hydrogen atom or is chosen from radicals (a1) to (a32): 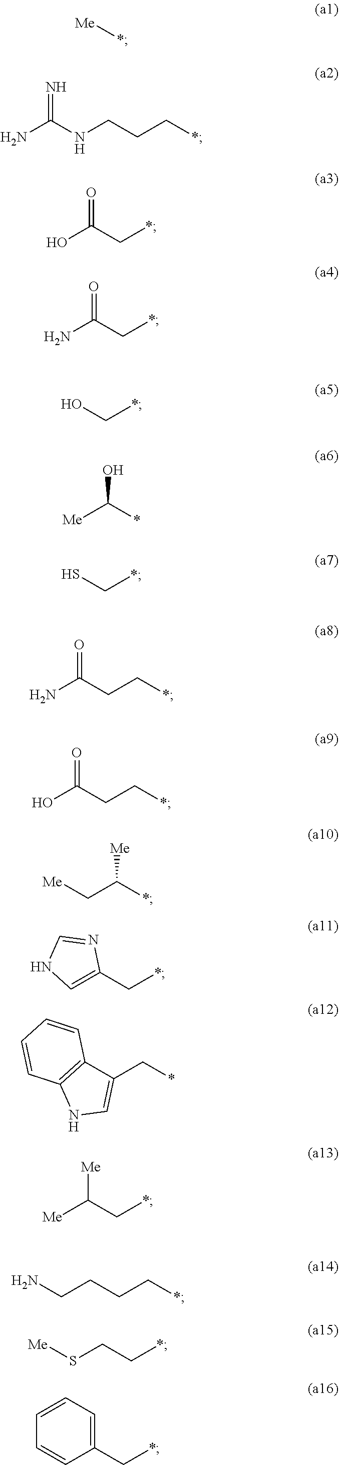 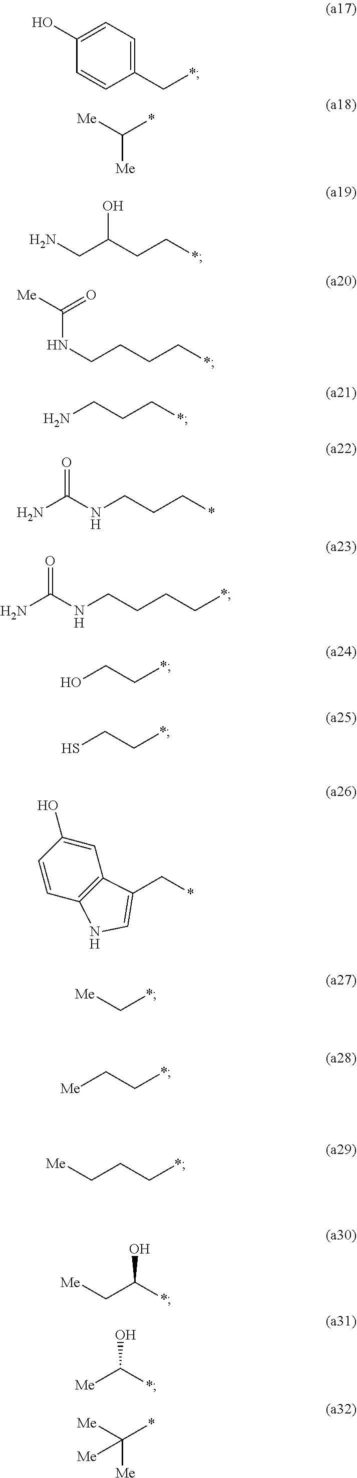
or, when i=0, Rₐ and R₄ form, together with the nitrogen atom which bears Rₐ and with the carbon atom which bears R₄, a saturated 5- or 6-membered heterocycle of formula A1, A2, or A3: 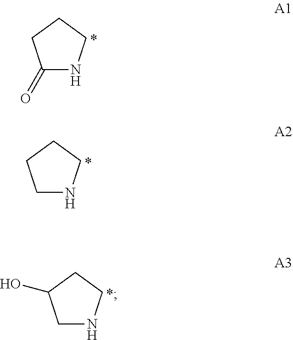 [SMILES: O=C1CCCN1 ; C1CCNC1 ; OC1CCNC1]
E) Rₐ is a hydrogen atom and R_{b} is a heterocycloalkyl radical, optionally substituted with at least one identical or different radical chosen from hydroxyl, C₁-C₄ hydroxyalkyl, C₁-C₄ alkyl, or C₁-C₄ alkoxy, and/or at least one of the ring members of the heterocycloalkyl radical is a ketone (—CO—); or
F) Rₐ is a hydrogen atom and R_{b} represents a glucopyran radical of formula (V): 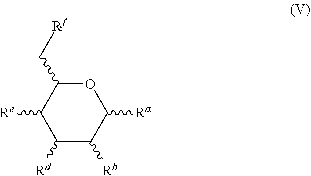
wherein in formula (V), Rₐ, R^{b}, R^{d}, R^{e}, and R^{f}, which are identical or different, are chosen from:
i) a hydroxyl group,
ii) (C₁-C₄)alkoxy, the alkyl part of which is optionally substituted with at least one hydroxyl group,
iii) carboxy, or
iv) an NR₁R₂ group, wherein R₁ and R₂, which are identical or different, are chosen from a hydrogen atom, (C₁-C₄)alkyl, or acetyl; and
wherein one of the radicals Rₐ, R^{b}, R^{d}, R^{e}, and R^{f} represents a covalent bond with the nitrogen atom of the NRₐR_{b} radical; and the configuration of the glucopyran radical of formula (V) is D or Land of α (alpha) or β (beta) anomeric configuration.

23. The method of claim 18, wherein the at least of one compound of formula is chosen from compounds 1′ to 15′ and 18′ to 20′, salts thereof, solvates thereof, optical and/or geometrical isomers thereof, including enantiomers and diastereoisomers thereof, racemates thereof, or mixtures thereof:

| Structure | Compound |
|---|---|
| 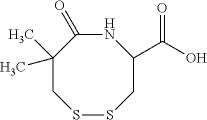 | 1′ |
| 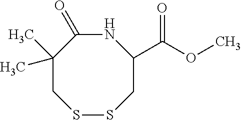 | 2′ |
| 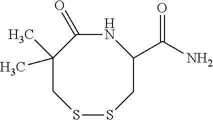 | 3′ |
| 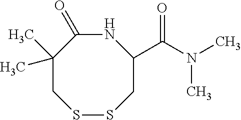 | 4′ |
| 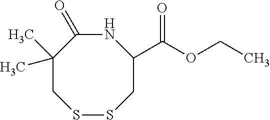 [SMILES: CCOC(=O)C1CSSCC(C)(C)C(=O)N1] | 5′ |
| 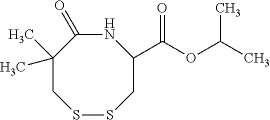 [SMILES: CC(C)OC(=O)C1CSSCC(C)(C)C(=O)N1] | 6′ |
| 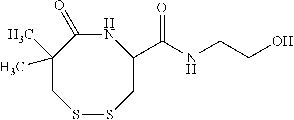 [SMILES: CC1(C)CSSCC(C(=O)NCCO)NC1=O] | 7′ |
| 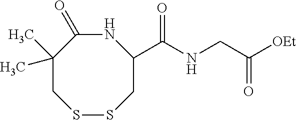 [SMILES: CCOC(=O)CNC(=O)C1CSSCC(C)(C)C(=O)N1] | 8′ |
| 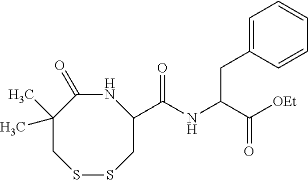 [SMILES: CCOC(=O)C(Cc1ccccc1)NC(=O)C1CSSCC(C)(C)C(=O)N1] | 9′ |
| 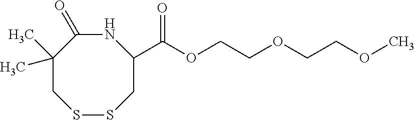 | 10′ |
| 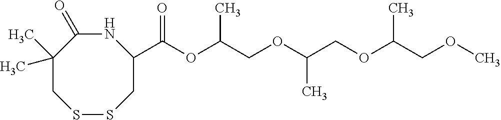 | 11′ |
| 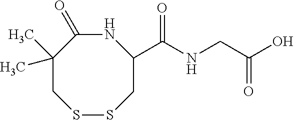 [SMILES: CC1(C)CSSCC(C(=O)NCC(=O)O)NC1=O] | 12′ |
| 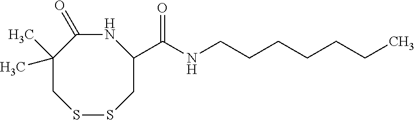 [SMILES: CCCCCCCNC(=O)C1CSSCC(C)(C)C(=O)N1] | 13′ |
| 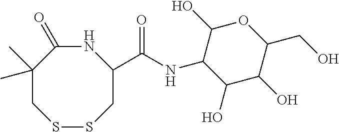 [SMILES: CC1(C)CSSCC(C(=O)NC2C(O)OC(CO)C(O)C2O)NC1=O] | 14′ |
| 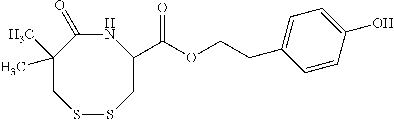 [SMILES: CC1(C)CSSCC(C(=O)OCCc2ccc(O)cc2)NC1=O] | 15′ |
| 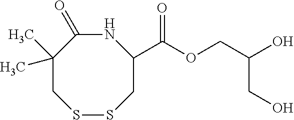 [SMILES: CC1(C)CSSCC(C(=O)OCC(O)CO)NC1=O] | 18′ |
| 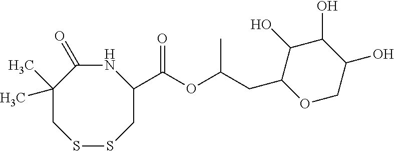 [SMILES: CC(CC1OCC(O)C(O)C1O)OC(=O)C1CSSCC(C)(C)C(=O)N1] | 19′ |
| 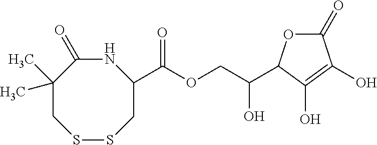 [SMILES: CC1(C)CSSCC(C(=O)OCC(O)C2OC(=O)C(O)=C2O)NC1=O] | 20′ |

24. The method of claim 23, wherein the at least one compound of formula (I) is chosen from the compounds 5′ to 15′ and 18′ to 20′, or compounds 1 to 4 below, salts thereof, solvates thereof, optical and/or geometrical isomers thereof, including enantiomers and diastereoisomers thereof, racemates thereof, or mixtures thereof:
7,7-dimethyl-6-oxo-1,2,5-dithiazocane-4-carboxylic acid (compound 1 (R enantiomer)),
methyl 7,7-dimethyl-6-oxo-1,2,5-dithiazocane-4-carboxylate (compound 2),
7,7-dimethyl-6-oxo-1,2,5-dithiazocane-4-carboxamide (compound 3),
N,N,7,7-tetramethyl-6-oxo-1,2,5-dithiazocane-4-carboxamide (compound 4),

| Structure | Compound |
|---|---|
| 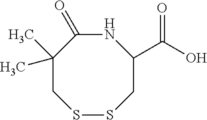 | 1 (R enantiomer) |
| 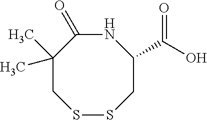 | |
| 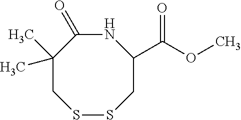 | 2 |
| 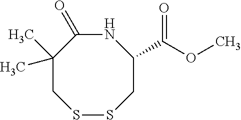 | |
| 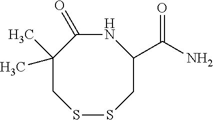 | 3 |
| 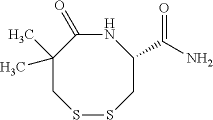 | |
| 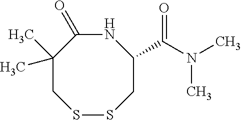 | 4 |
| 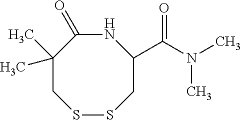 | |

25. The method of claim 18, wherein in the at least one compound of formula (I) is chosen from 7,7-dimethyl-6-oxo-1,2,5-dithiazocane-4-(R)-carboxylic acid (compound 1), methyl 7,7-dimethyl-6-oxo-1,2,5-dithiazocane-4 (R)-carboxylate (compound 2) below:

| Compound | Structure |
|---|---|
| 1 | 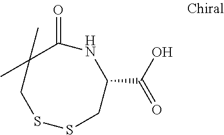 |
| 2 | 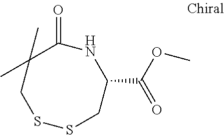 |

salts thereof, solvates thereof, optical and/or geometrical isomers thereof, including enantiomers and diastereoisomers thereof, racemates thereof, or mixtures thereof.

26. The method of claim 18, where the method comprises topically applying the composition to skin.

27. A compound of formula (I), a salt thereof, a solvate thereof, an optical isomer thereof, a geometrical isomer thereof, including an enantiomer or diastereoisomer thereof, or a racemate thereof, 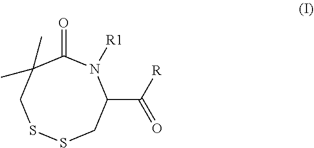
wherein in formula (I):
R is chosen from a radical OR′ or NRₐR_{b};
R₁ is chosen from a hydrogen atom or a (C₁-C₁₈)alkyl group;
R′ is chosen from:
i) a hydrogen atom;
ii) a (C₁-C₁₈)alkyl group, wherein:
a) the (C₁-C₁₈)alkyl group is optionally substituted with at least one identical or different radical chosen from:
hydroxyl;
(hetero)cycloalkyl, optionally substituted with at least one identical or different radical chosen from hydroxyl, C₁-C₄ alkyl, C₁-C₄ hydroxyalkyl, or C₁-C₄ alkoxy, and/or at least one of ring members of the (hetero)cycloalkyl denoting a ketone (—CO—); or
aryl, optionally substituted with at least one identical or different radical chosen from C₁-C₄ alkyl, C₁-C₄ alkoxy, or hydroxyl; and/or
b) the (C₁-C₁₈)alkyl group is optionally interrupted with at least one heteroatom;
iii) a (C₂-C₁₈)alkenyl group, wherein:
a) the (C₂-C₁₈)alkenyl group is optionally substituted with at least one identical or different radical chosen from:
hydroxyl; or
aryl, optionally substituted with at least one identical or different radical chosen from C₁-C₄ alkyl, C₁-C₄ alkoxy, or hydroxyl; and/or
b) the (C₂-C₁₈)alkenyl group is optionally interrupted with at least one heteroatom; or
iv) an optionally substituted aryl or heteroaryl radical;
Rₐ and R_{b} are independently chosen from:
i) a hydrogen atom;
ii) a (C₁-C₁₈)alkyl group, wherein:
a) the (C₁-C₁₈)alkyl group is optionally substituted with at least one identical or different radical chosen from:
hydroxyl;
carboxy;
(C₁-C₆)alkyloxycarbonyl; or
aryl, optionally substituted with at least one identical or different radical chosen from C₁-C₄ alkyl, C₁-C₄ alkoxy, or hydroxyl; and/or
b) the (C₁-C₁₈)alkyl group is optionally interrupted with at least one heteroatom;
iii) a (C₂-C₁₈)alkenyl group, wherein:
a) the (C₂-C₁₈)alkenyl group is optionally substituted with at least one identical or different radical chosen from:
hydroxyl;
carboxy;
(C₁-C₆)alkyloxycarbonyl; or
aryl; optionally substituted with at least one identical or different radical chosen from C₁-C₄ alkyl, C₁-C₄ alkoxy, or hydroxyl; and/or
b) the (C₂-C₁₈)alkenyl group is optionally interrupted with at least one heteroatom;
iv) an aryl or heteroaryl radical, optionally substituted with at least one identical or different radical chosen from C₁-C₄ alkyl, hydroxyl, or C₁-C₄ alkoxy; or
v) a heterocycloalkyl radical, optionally substituted with at least one identical or different radical chosen from hydroxyl, C₁-C₄ hydroxyalkyl, C₁-C₄ alkyl, or C₁-C₄ alkoxy, and/or at least one of ring members of the heterocycloalkyl radical denoting a ketone (—CO—); or
Ra and Rb form, together with the nitrogen atom to which Rₐ and R_{b} are attached, an optionally substituted heterocycloalkyl;
wherein the compound is not chosen from compounds Cp1 to Cp5 below:
1,2,5-Dithiazocine-4-carboxamide, hexahydro-7,7-dimethyl-6-oxo-, (4R)-(Cp1),
1,2,5-Dithiazocine-4-carboxamide, hexahydro-N,N,7,7-tetramethyl-6-oxo-, (4R)-Rotation (−) (Cp2),
1,2,5-Dithiazocine-4-carboxylic acid, hexahydro-7,7-dimethyl-6-oxo-, methyl ester, (R)-(9Cl) (Cp3),
7,7-dimethyl-6-oxo-1,2,5-dithiazocane-4-carboxylic acid (Cp4),

| Structure | Compound |
|---|---|
| 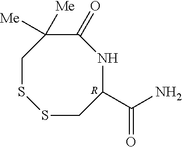 | Cp1 |
| 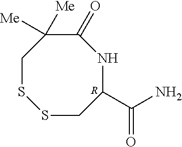 | Cp2 |
| 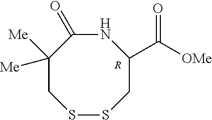 | Cp3 |
| 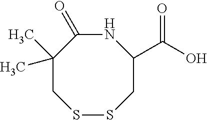 | Cp4 |

or salts thereof, solvates thereof, optical isomers thereof, geometrical isomers thereof, including enantiomers and diastereoisomers, and racemates thereof, or mixtures thereof.

28. The compound of claim 27, wherein the compound is chosen from compounds of formula (F1), salts thereof, solvates thereof, optical isomers thereof, geometrical isomers thereof, including enantiomers and diastereoisomers thereof, racemates thereof, or mixtures thereof: 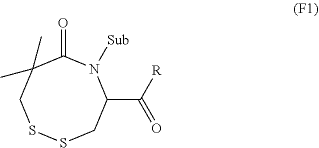
wherein in formula (F1):
R is chosen from a radical OR′ or NRₐR_{b}; and
Sub represents a (C₁-C₁₈)alkyl group.

29. The compound of claim 27, wherein the compound is chosen from compounds of formula (F2), salts thereof, solvates thereof, optical isomers thereof, geometrical isomers thereof, including enantiomers and diastereoisomers thereof, racemates thereof, or mixtures thereof: 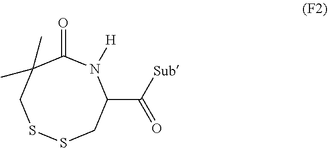
wherein in formula (F2):
Sub′ is a radical OT′ or NTₐT_{b};
T′ is chosen from:
i) a (C₂-C₁₈)alkyl group, wherein:
the (C₂-C₁₈)alkyl group is optionally substituted with at least one identical or different radical chosen from:
hydroxyl;
(hetero)cycloalkyl, optionally substituted with at least one identical or different radical chosen from hydroxyl, C₁-C₄ alkyl, C₁-C₄ hydroxyalkyl, or C₁-C₄ alkoxy, and/or at least one of the ring members of the (hetero)cycloalkyl denoting a ketone (—CO—);
aryl, optionally substituted with at least one identical or different radical chosen from C₁-C₄ alkyl, C₁-C₄ alkoxy, or hydroxyl; and/or
the (C₂-C₁₈)alkyl group is optionally interrupted with at least one heteroatom;
ii) a (C₂-C₁₈)alkenyl group, wherein:
the (C₂-C₁₈)alkenyl group is optionally substituted with at least one identical or different radical chosen from:
hydroxyl,
aryl, optionally substituted with at least one identical or different radical chosen from C₁-C₄ alkyl, C₁-C₄ alkoxy, or hydroxyl; and/or
the (C₂-C₁₈)alkenyl group is optionally interrupted with at least one heteroatom; or
iii) an optionally substituted aryl or heteroaryl radical;
Ta represents a radical chosen from:
i) a hydrogen atom;
ii) a (C₂-C₁₈)alkyl group,
the (C₂-C₁₈)alkyl group being optionally substituted with at least one identical or different radicals chosen from:
hydroxyl,
carboxy,
(C₁-C₆)alkyloxycarbonyl,
aryl, optionally substituted with at least one identical or different radical chosen from C₁-C₄ alkyl, C₁-C₄ alkoxy, or hydroxyl; and/or
the (C₂-C₁₈)alkyl group is optionally interrupted with at least one heteroatom;
iii) a (C₂-C₁₈)alkenyl group,
the (C₂-C₁₈)alkenyl group being optionally substituted with at least one identical or different radicals chosen from:
hydroxyl,
carboxy,
(C₁-C₆)alkyloxycarbonyl,
aryl, optionally substituted with at least one identical or different radical chosen from C₁-C₄ alkyl, C₁-C₄ alkoxy, or hydroxyl; and/or
the (C₂-C₁₈)alkenyl group is optionally interrupted with at least one heteroatom;
iv) an aryl or heteroaryl radical, optionally substituted with at least one identical or different radical chosen from C₁-C₄ alkyl, hydroxyl, or C₁-C₄ alkoxy;
v) a heterocycloalkyl radical, optionally substituted with at least one identical or different radical chosen from hydroxyl, C₁-C₄ hydroxyalkyl, C₁-C₄ alkyl, or C₁-C₄ alkoxy, and/or at least one of the ring members of the heterocycloalkyl radical denoting a ketone (—CO—);
T_{b} represents a radical chosen from:
i) a hydrogen atom;
ii) a (C₁-C₁₈)alkyl group,
the (C₁-C₁₈)alkyl group being optionally substituted with at least one identical or different radical chosen from:
hydroxyl,
carboxy,
(C₁-C₆)alkyloxycarbonyl,
aryl, optionally substituted with at least one identical or different radicals chosen from C₁-C₄ alkyl, C₁-C₄ alkoxy, hydroxyl, and/or
the (C₁-C₁₈)alkyl group is optionally interrupted with at least one heteroatom;
iii) a (C₂-C₁₈)alkenyl group,
the (C₂-C₁₈)alkenyl group being optionally substituted with at least one identical or different radicals chosen from:
hydroxyl,
carboxy,
(C₁-C₆)alkyloxycarbonyl,
aryl, optionally substituted with at least one identical or different radical chosen from C₁-C₄ alkyl, C₁-C₄ alkoxy, or hydroxyl; and/or
the (C₂-C₁₈)alkenyl group is optionally interrupted with at least one heteroatom;
iv) an aryl or heteroaryl radical, optionally substituted with at least one identical or different radical chosen from C₁-C₄ alkyl, hydroxyl, or C₁-C₄ alkoxy;
v) a heterocycloalkyl radical, optionally substituted with at least one identical or different radical chosen from hydroxyl, C₁-C₄ hydroxyalkyl, C₁-C₄ alkyl, or C₁-C₄ alkoxy, and/or at least one of the ring members of the heterocycloalkyl radical denoting a ketone (—CO—); or
Ta and T_{b} form, together with the nitrogen atom to which Ta and T_{b} are attached, an optionally substituted heterocycloalkyl.

30. The compound of claim 27, wherein the compound is chosen from compounds 5′ to 15′ and 18′ to 20′ below:
ethyl 7,7-dimethyl-6-oxo-1,2,5-dithiazocane-4-carboxylate (compound 5′);
propan-2-yl 7,7-dimethyl-6-oxo-1,2,5-dithiazocane-4-carboxylate (compound 6′);
N-(2-hydroxyethyl)-7,7-dimethyl-6-oxo-1,2,5-dithiazocane-4-carboxamide (compound 7′);
ethyl N-[(7,7-dimethyl-6-oxo-1,2,5-dithiazocan-4-yl)carbonyl]glycinate (compound 8′);
ethyl N-[(7,7-dimethyl-6-oxo-1,2,5-dithiazocan-4-yl)carbonyl]phenylalaninate (compound 9′);
2-(2-methoxyethoxy)ethyl 7,7-dimethyl-6-oxo-1,2,5-dithiazocane-4-carboxylate (compound 10′);
1-({1-[(1-methoxypropan-2-yl)oxy]propan-2-yl}oxy)propan-2-yl 7,7-dimethyl-6-oxo-1,2,5-dithiazocane-4-carboxylate (compound 11′);
N-[(7,7-dimethyl-6-oxo-1,2,5-dithiazocan-4-yl)carbonyl]glycine (compound 12′);
N-heptyl-7,7-dimethyl-6-oxo-1,2,5-dithiazocane-4-carboxamide (compound 13′);
2-deoxy-2-{[(7,7-dimethyl-6-oxo-1,2,5-dithiazocan-4-yl)carbonyl]amino}hexo-pyranose (compound 14′);
2-(4-hydroxyphenyl)ethyl 7,7-dimethyl-6-oxo-1,2,5-dithiazocane-4-carboxylate (compound 15′);
2,3-dihydroxypropyl 7,7-dimethyl-6-oxo-1,2,5-dithiazocane-4-carboxylate (compound 18′);
4,8-anhydro-1,3-dideoxy-2-O-[(7,7-dimethyl-6-oxo-1,2,5-dithiazocan-4-yl)-carbonyl]octitol (compound 19′); or
2-(3,4-dihydroxy-5-oxo-2,5-dihydrofuran-2-yl)-2-hydroxyethyl 7,7-dimethyl-6-oxo-1,2,5-dithiazocane-4-carboxylate (compound 20′):

| Structure | Compound |
|---|---|
| 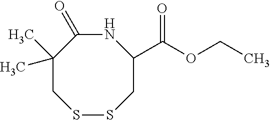 [SMILES: CCOC(=O)C1CSSCC(C)(C)C(=O)N1] | 5′ |
| 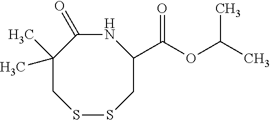 [SMILES: CC(C)OC(=O)C1CSSCC(C)(C)C(=O)N1] | 6′ |
| 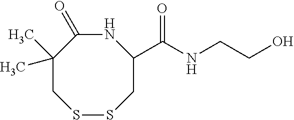 [SMILES: CC1(C)CSSCC(C(=O)NCCO)NC1=O] | 7′ |
| 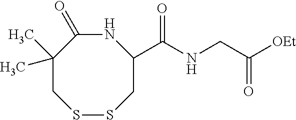 [SMILES: CCOC(=O)CNC(=O)C1CSSCC(C)(C)C(=O)N1] | 8′ |
| 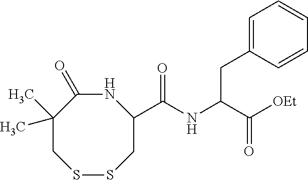 [SMILES: CCOC(=O)C(Cc1ccccc1)NC(=O)C1CSSCC(C)(C)C(=O)N1] | 9′ |
| 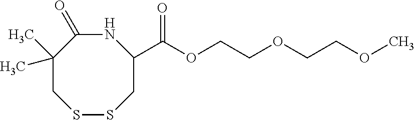 | 10′ |
| 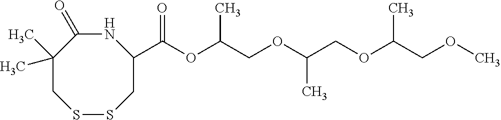 | 11′ |
| 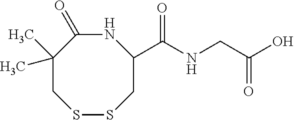 [SMILES: CC1(C)CSSCC(C(=O)NCC(=O)O)NC1=O] | 12′ |
| 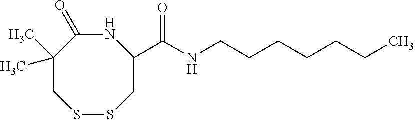 [SMILES: CCCCCCCNC(=O)C1CSSCC(C)(C)C(=O)N1] | 13′ |
| 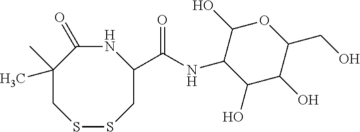 [SMILES: CC1(C)CSSCC(C(=O)NC2C(O)OC(CO)C(O)C2O)NC1=O] | 14′ |
| 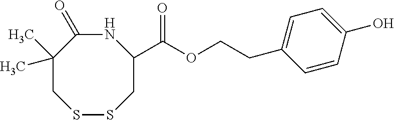 [SMILES: CC1(C)CSSCC(C(=O)OCCc2ccc(O)cc2)NC1=O] | 15′ |
| 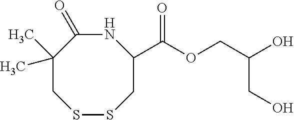 [SMILES: CC1(C)CSSCC(C(=O)OCC(O)CO)NC1=O] | 18′ |
| 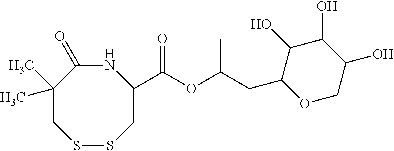 [SMILES: CC(CC1OCC(O)C(O)C1O)OC(=O)C1CSSCC(C)(C)C(=O)N1] | 19′ |
| 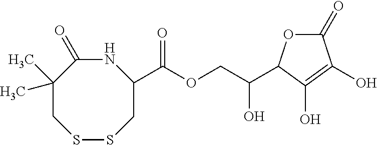 [SMILES: CC1(C)CSSCC(C(=O)OCC(O)C2OC(=O)C(O)=C2O)NC1=O] | 20′ |

salts thereof, solvates thereof, optical isomers thereof, geometrical isomers thereof, including enantiomers and diastereoisomers thereof, racemates thereof, or mixtures thereof.

31. The compound of claim 27, wherein the compound is chosen from 7,7-dimethyl-6-oxo-1,2,5-dithiazocane-4-(R)-carboxylic acid (compound 1), methyl 7,7-dimethyl-6-oxo-1,2,5-dithiazocane-4 (R)-carboxylate (compound 2) below:

| Compound | Structure |
|---|---|
| 1 | 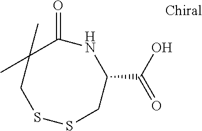 |
| 2 | 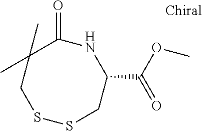 |

salts thereof, solvates thereof, optical and/or geometrical isomers thereof, including enantiomers and diastereoisomers thereof, racemates thereof, or mixtures thereof.

32. A composition comprising, in a physiologically acceptable medium, at least one compound of formula (I), 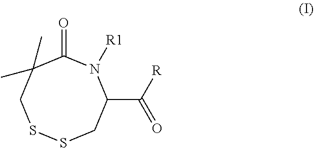
a salt thereof, a solvate thereof, an optical isomer thereof, a geometrical isomer thereof, including an enantiomer and a diastereoisomer thereof, a racemate thereof, or a mixture thereof;
wherein in formula (I):
R is chosen from a radical OR′ or NRₐR_{b};
R₁ is chosen from a hydrogen atom or a (C₁-C₁₈)alkyl group;
R′ is chosen from:
i) a hydrogen atom;
ii) a (C₁-C₁₈)alkyl group, wherein:
a) the (C₁-C₁₈)alkyl group is optionally substituted with at least one identical or different radical chosen from:
hydroxyl;
(hetero)cycloalkyl, optionally substituted with at least one identical or different radical chosen from hydroxyl, C₁-C₄ alkyl, C₁-C₄ hydroxyalkyl, or C₁-C₄ alkoxy, and/or at least one of ring members of the (hetero)cycloalkyl denoting a ketone (—CO—); or
aryl, optionally substituted with at least one identical or different radical chosen from C₁-C₄ alkyl, C₁-C₄ alkoxy, or hydroxyl; and/or
b) the (C₁-C₁₈)alkyl group is optionally interrupted with at least one heteroatom;
iii) a (C₂-C₁₈)alkenyl group, wherein:
a) the (C₂-C₁₈)alkenyl group is optionally substituted with at least one identical or different radical chosen from:
hydroxyl; or
aryl, optionally substituted with at least one identical or different radical chosen from C₁-C₄ alkyl, C₁-C₄ alkoxy, or hydroxyl; and/or
b) the (C₂-C₁₈)alkenyl group is optionally interrupted with at least one heteroatom; or
iv) an optionally substituted aryl or heteroaryl radical;
Rₐ and R_{b} are independently chosen from:
i) a hydrogen atom;
ii) a (C₁-C₁₈)alkyl group, wherein:
a) the (C₁-C₁₈)alkyl group is optionally substituted with at least one identical or different radical chosen from:
hydroxyl;
carboxy;
(C₁-C₆)alkyloxycarbonyl; or
aryl, optionally substituted with at least one identical or different radical chosen from C₁-C₄ alkyl, C₁-C₄ alkoxy, or hydroxyl; and/or
b) the (C₁-C₁₈)alkyl group is optionally interrupted with at least one heteroatom;
iii) a (C₂-C₁₈)alkenyl group, wherein:
a) the (C₂-C₁₈)alkenyl group is optionally substituted with at least one identical or different radical chosen from:
hydroxyl;
carboxy;
(C₁-C₆)alkyloxycarbonyl; or
aryl; optionally substituted with at least one identical or different radical chosen from C₁-C₄ alkyl, C₁-C₄ alkoxy, or hydroxyl; and/or
b) the (C₂-C₁₈)alkenyl group is optionally interrupted with at least one heteroatom;
iv) an aryl or heteroaryl radical, optionally substituted with at least one identical or different radical chosen from C₁-C₄ alkyl, hydroxyl, or C₁-C₄ alkoxy; or
v) a heterocycloalkyl radical, optionally substituted with at least one identical or different radical chosen from hydroxyl, C₁-C₄ hydroxyalkyl, C₁-C₄ alkyl, or C₁-C₄ alkoxy, and/or at least one of ring members of the heterocycloalkyl radical denoting a ketone (—CO—); or
Rₐ and R_{b} form, together with the nitrogen atom to which Rₐ and R_{b} are attached, an optionally substituted heterocycloalkyl.

33. The composition of claim 32, wherein the at least one compound of formula (I) is present in an amount of between 0.01% and 10% by weight, relative to the total weight of the composition.

34. The composition of claim 32, wherein the at least one compound of formula (I) is present in an amount of between 0.1% and 5% by weight, relative to the total weight of the composition.

35. The composition of claim 32, wherein the at least one compound of formula (I) is present in an amount of between 0.5% and 3% by weight, relative to the total weight of the composition.

36. The composition of claim 32, wherein the composition further comprises at least one adjuvant chosen from organic solvents, oils, waxes, pigments, fillers, dyes, surfactants, emulsifiers, cosmetic active agents, organic or mineral photoprotective agents, polymers, thickeners, preservatives, fragrances, bactericides, ceramides, odor absorbers, or antioxidants.
